(19) United States
(12) Patent Application Publication
(10) Pub. No.: US 2009/0048227 A1
(43) Pub. Date: Feb. 19, 2009

(54) Substituted-1-Phthalazinamines As Vr- 1 Antagonists

(76) Inventors: Pasun K. Chakravarty, Edison, NJ (US); Pengcheng Patrick Shao, Fanwood, NJ (US); Feng Ye, Scotch Plains, NJ (US)

Correspondence Address: MERCK AND CO., INC, P O BOX 2000, RAHWAY, NJ, 07065-0907, US

(21) Appl. No.: 12/086,740

(22) PCT Filed: Dec. 18, 2006
(86) PCT No.: PCT/US2006/048162

§ 371 Date: Jun. 17, 2008

Related U.S. Application Data
(60) Provisional application No. 60/753,998, filed on Dec. 22, 2005.

(51) Int. Cl. A61K 31/397; C07D 401/02; C07D 471/04; C07D 211/58; A61K 31/497; A61K 31/4545; A61K 31/4535; A61K 31/4525; A61P 29/00; A61K 31/453; A61K 31/454; A61K 31/4468; C07D 405/02; C07D 413/02; C07D 409/02

(52) U.S. Cl. 514/210.18; 546/223; 546/194; 546/214; 546/208; 544/336; 544/408; 546/209; 546/172; 546/207; 546/211; 546/121; 514/255.05; 514/300; 514/314; 514/318; 514/322; 514/326; 514/329

(57) ABSTRACT

Substituted piperidine compounds represented by Formula I, or pharmaceutically acceptable salts thereof. Pharmaceutical compositions comprise an effective amount of the instant compounds, either alone, or in combination with one or more other therapeutically active compounds, and a pharmaceutically acceptable carrier. Methods of treating conditions associated with, or caused by, sodium channel activity, including, for example, acute pain, chronic pain, visceral pain, inflammatory pain, neuropathic pain, urinary incontinence, itchiness, allergic dermatitis, epilepsy, irritable bowel syndrome, depression, anxiety, multiple sclerosis, and bipolar disorder, comprise administering an effective amount of the present compounds, either alone, or in combination with one or more other therapeutically active compounds.

### FIELD OF THE INVENTION

This invention relates to substituted piperidine compounds. In particular, this invention relates to substituted piperidine compounds that are N-type calcium channel blockers useful for the treatment of a variety of pain conditions including chronic and neuropathic pain. The compounds of the present invention are also useful for the treatment of other conditions, including disorders of bladder function, pruritis, itchiness, allergic dermatitis and disorders of the central nervous system (CNS) such as stroke, epilepsy, manic depression, bipolar disorder, depression, anxiety and diabetic neuropathy.

### BACKGROUND TO THE INVENTION

Ion channels control a wide range of cellular activities in both excitable and non-excitable cells (Hille, 2002). Ion channels are attractive therapeutic targets due to their involvement in many physiological processes. In excitable cells, the coordinated function of the resident set of ion channels controls the electrical behavior of the cell. Voltage-gated calcium channels provide an important link between electrical activity at the plasma membrane and cell activities that are dependent on intracellular calcium, including muscle contraction, neurotransmitter release, hormone secretion and gene expression. Voltage-gated calcium channels serve to integrate and transduce plasma membrane electrical activity into changes in intracellular calcium concentration, and can do this on a rapid time scale.

Because of this crucial role in cell physiology, modulation of calcium channel activity can have profound effects. Mutations in calcium channel subunits have been implicated in a number of genetic diseases including familial hemiplegic migraine, spinocerebellar ataxia, Timothy Syndrome, incomplete congenital stationary night blindness and familial hypokalemic periodic paralysis. Modulation of voltage-gated calcium channels by signaling pathways, including c-AMP-dependent protein kinases and G proteins is an important component of signaling by hormones and neurotransmitters (Catterall, 2000). Pharmacological modulation of calcium channels can have significant therapeutic effects, including the use of L-type calcium channel (Cav1.2) blockers in the treatment of hypertension (Hockerman, et al., 1997) and more recently, use of Ziconitide, a peptide blocker of N-type calcium channels (Cav2.2), for the treatment of intractable pain (Staats, et al., 2004). Zicontide is derived from Conotoxin, a peptide toxin isolated from cone snail venom. Ziconitide must be applied by intrathecal injection to allow its access to a site of action in the spinal cord and to minimize exposure to channels in the autonomic nervous system that are involved in regulating cardiovascular function. Ziconotide has also been shown to highly effective as a neuroprotective agent in rat models of global and focal ischemia (Colburne et. Al., Stroke (1999) 30, 662-668) suggesting that modulation of N-type calcium channels (Cav2.2) has implication in the treatment of stroke.

Clinical and preclinical experiments with ziconitide and related peptides confirm a key role of N-type calcium channels in transmitting nociceptive signals into the spinal cord. Identification of N-type calcium channel blockers that can be administered systemically, and effectively block N-type calcium channels in the nociceptive signaling pathway, while sparing N-type calcium channel function in the periphery would provide important new tools for treating some forms of pain. The present invention describes blockers of N-type calcium channels (Cav2.2) that display function selectivity by blocking N-type calcium channel activity needed to maintain pathological nociceptive signaling, while exhibiting a lesser potency at blocking N-type calcium channels involved in maintaining normal cardiovascular function.

### SUMMARY OF THE INVENTION

The present invention is directed to series of substituted piperidine compounds which are N-type calcium channel (Cav2.2) blockers useful for the treatment of acute pain, chronic pain, cancer pain, visceral pain, inflammatory pain, neuropathic pain, post-herpetic neuralgia, diabatic neuropathy, trigeminal neuralgia, migrane, fibromyalgia and stroke. The compounds of the present invention are also useful for the treatment of other conditions, including disorders of bladder function, pruritis, itchiness, allergic dermatitis, and disorders of the CNS such as anxiety, depression, epilepsy, manic depression and bipolar disorder. This invention also provides pharmaceutical compositions comprising a compound of the present invention, either alone, or in combination with one or more therapeutically active compounds, and a pharmaceutically acceptable carrier.

This invention further comprises methods for the treatment of acute pain, chronic pain, visceral pain, inflammatory pain, neuropathic pain and disorders of the CNS including, but not limited to, epilepsy, manic depression, depression, anxiety and bipolar disorder comprising administering the compounds and pharmaceutical compositions of the present invention.

### DETAILED DESCRIPTION OF THE INVENTION

The compounds of this invention are represented by Formula I:

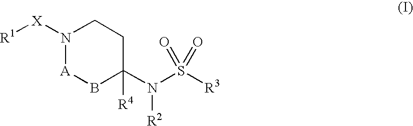

or pharmaceutically acceptable salts thereof, wherein:

### X is —CH(R^{a})—, C═O, (C═O)O, NH(C═O), SO₂ or —NHSO₂;

A and B each independently is CH₂, CH—C₁-C₄alkyl, CH—OH or CO;

### R¹ is:

(a) C₁-C₈ alkyl,
(b) C₃-C₆ cycloalkyl,
(c) C₃-C₆ cycloalkyl-C(O)O—C₀-C₆alkyl,
(d) aryl-heteroaryl,

### (e) N-heterocycle,

### (f) N-aryl,

(g) C₁-C₄ alkyl-COOH,
(h) C₁-C₄alkl-C(O)—N—C₁-C₄alkyl-R^{a},
(i) N—C₁-C₄ alkyl(aryl)(COOH),
(j) C₁-C₄alkyl(N—C(O)-heterocycle)(C₀-C₄alkyl-aryl),
(k) C₁-C₄alkyl(N—C(O)O—C₁-C₄alkyl)(C₀-C₄-alkyl-C₀-C₄ perfluoroalkyl),
(l) C₁-C₄alkyl-N—C(O)-aryl,
(m) C₁-C₄alkyl-N—C(O)—C₃-C₆ cycloakyl, or

### (n) O—R^{a}

(o) C₀-C₄ alkyl-aryl, where said aryl is substituted with one or more substituents selected from heteroaryl, N—C₁-C₄alkyl-C(O)R^{a}, C(O)—N—C₁-C₄alkyl-W, SO₂R^{a}, C(O)R^{a}, C(O)O—R^{a}, and optionally substituted with one or more substituents selected from halogen, aryl, C₁-C₆ alkyl, C₁-C₆ haloalkyl, alkoxy, NR^{a}, O—CF₃, CN, C₃-C₆ cycloalkyl, or OH;
(p) C₀-C₄ alkyl-heteroaryl, where said heteroaryl is substituted with one or more substituents selected from heteroaryl, N—C₁-C₄alkyl-C(O)R⁸, C(O)—N—C₁-C₄alkyl-R^{a}, SO₂R^{a}, C(O)R^{a}, C(O)O—R^{a}, and optionally substituted with one or more substituents selected from halogen, aryl, C₁-C₆ alkyl, C₁-C₆ haloalkyl, alkoxy, NR^{a}, O—CF₃, CN, C₃-C₆ cycloalkyl, or OH;

### R² is

### (a) H,

(b) C₁-C₆-alkyl,

### (c) C₁-C₆—OH,

(d) C₃-C₆ cycloalkyl, or
(e) C₀-C₆ alkyl aryl, wherein said aryl is optionally substituted with one or more substituents selected from alkyl, O—C₁-C₄ alkyl, and halogen,
or the N to which R² is attached can join with two adjacent atoms to form a heterocycle, or N and the SO₂ to which it is attached can join to form a heterocycle, wherein said heterocycle is optionally substituted with one or more substituents selected from O and CF₃,

### R³ is:

(a) aryl, or
(b) heteroaryl,
said aryl is optionally substituted with one or more substituents selected from CF₃, CN, halogen, C₁-C₄—OH, N—R^{a}, O—R^{a}, and N—C(O)—R^{a}, and said heteroaryl is optionally substituted with one or more substituents selected from S—R^{a}, heterocycle, and C(O)—N—R^{a};

### R⁴ is:

### (a) H,

(b) —C₁-C₄-alkyl, or

### (c) OH;

### R^{a} is:

### (a) H,

(b) C₁-C₆ alky,
(c) —C₀-C₆-alkyl-heterocycloalkyl,
(d) —C₁-C₆-alkoxy,

### (e) NH₂, or

(f) —C₀-C₆-aryl.

### R^{b} is:

### (a) H,

(b) C₁-C₆ alkyl,

### (c) OH,

(d) —C₁-C₆-alkoxy,

### (e) NH₂, or

(f) NH—C₁-C₄-alkyl or N(C₁-C₄-alkyl)₂.

A first embodiment of the present invention includes compounds wherein X is C═O.

A second embodiment of the present invention includes compounds wherein X is CH₂.

A third embodiment of the present invention includes compounds wherein X is SO₂.

A fourth embodiment of the present invention includes compounds wherein X is —CO—NH—.

A fifth embodiment of the present invention includes compounds wherein A and B each independently is CH₂.

A sixth embodiment of the present invention includes compounds wherein A is CO and B is CH₂.

A seventh embodiment of the present invention includes compounds wherein A is CH₂ and B is CO.

An eighth embodiment of the present invention includes compounds wherein A is CH₂ and B is CH(OH).

A ninth embodiment of the present invention includes compounds wherein R¹ is phenyl substituted with one or more substituents selected from SO₂—R^{a}, SO₂NH₂, CONH₂, CONHCH₃, and COOCH₃.

A tenth embodiment of the present invention includes compounds wherein R² is:

(1) hydrogen, or

(2) C₃-C₆ cycloalkyl.

An eleventh embodiment of the present invention includes compounds wherein R² is cyclopropyl.

A twelfth embodiment of the present invention includes compounds wherein R³ is phenyl.

A thirteenth embodiment of the present invention includes compounds wherein R³ is phenyl substituted with one or more CF₃.

A fourteenth embodiment of the present invention includes compounds wherein R⁴ is hydrogen.

Additional embodiments of the present invention include compounds of the Formula Ia:

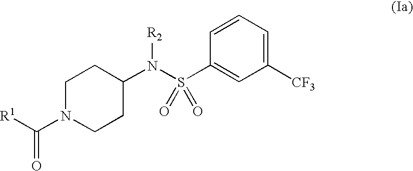

or a pharmaceutically acceptable salt thereof, wherein R¹ and R² are as defined in Formula I.

Further embodiments of the present invention include compounds of the Formula Ib:

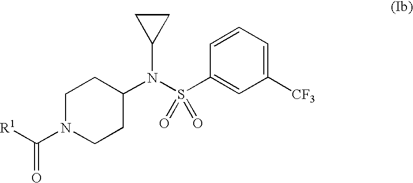

or a pharmaceutically acceptable salt thereof, wherein R¹ is as defined in Formula I.

Further embodiments of the present invention include compounds of the Formula Ic:

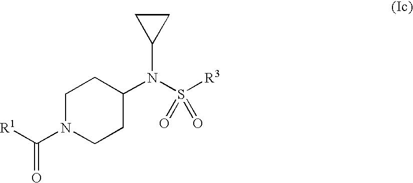

or a pharmaceutically acceptable salt thereof, wherein R¹ is as defined in Formula I and R³ is phenyl optionally substituted with one or more substituents selected from halogen, CF₃ and O—R^{a}.

Still further embodiments of the present invention include compounds of the Formula Id:

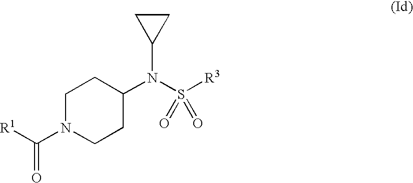

or a pharmaceutically acceptable salt thereof, wherein R³ is as defined in Formula I, and R¹ is C₁-C₆ alkyl, C₀-C₄ alkyl-aryl, or heteroaryl, wherein said aryl and heteroaryl each is independently optionally substituted with one or more substituents selected from SO₂—R^{a}, SO₂NH₂, CONH₂, CONHCH₃, and COOCH₃.

Still further embodiments of the present invention include compounds of the Formula Ie:

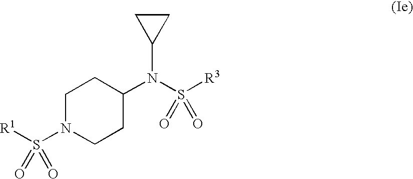

or a pharmaceutically acceptable salt thereof, wherein R³ is as defined in Formula I, and R¹ is C₀-C₄ alkyl-aryl, wherein said aryl is optionally substituted with one or more substituents selected from SO₂—R¹, SO₂NH₂, CONH₂, CONHCH₃, and COOCH₃.

As used herein, “alkyl” as well as other groups having the prefix “alk” such as, for example, alkoxy, alkanoyl, alkenyl, and alkynyl means carbon chains which may be linear or branched or combinations thereof. Examples of alkyl groups include methyl, ethyl, propyl, isopropyl, butyl, sec- and tert-butyl, pentyl, hexyl, and heptyl. “Alkenyl,” “alkynyl” and other like terms include carbon chains containing at least one unsaturated C—C bond.

The term “cycloalkyl” refers to a saturated hydrocarbon containing one ring having a specified number of carbon atoms. Examples of cycloalkyl include cyclopropyl, cyclobutyl, cyclopentyl, and cyclohexyl.

The term “C₀₋₄alkyl” includes alkyls containing 4, 3, 2, 1, or no carbon atoms. An alkyl with no carbon atoms is a hydrogen atom substituent when the alkyl is a terminal group and is a direct bond when the alkyl is a bridging group.

The term “alkoxy” as used herein, alone or in combination, includes an alkyl group connected to the oxy connecting atom. The term “alkoxy” also includes alkyl ether groups, where the term ‘alkyl’ is defined above, and ‘ether’ means two alkyl groups with an oxygen atom between them. Examples of suitable alkoxy groups include methoxy, ethoxy, n-propoxy, i-propoxy, n-butoxy, s-butoxy, t-butoxy, methoxymethane (also referred to as ‘dimethyl ether’), and methoxyethane (also referred to as ‘ethyl methyl ether’).

As used herein, “aryl” is intended to mean any stable monocyclic or bicyclic carbon ring of up to 7 members in each ring, wherein at least one ring is aromatic. Examples of such aryl elements include phenyl, napthyl, tetrahydronapthyl, indanyl, or biphenyl.

The term “heterocycle” or “heterocyclic”, as used herein except where noted, represents a stable 5- to 7-membered monocyclic- or stable 8- to 11-membered bicyclic heterocyclic ring system which is either saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Heterocycle includes bicyclic ring systems where one ring is aromatic and the other is not. Examples of heterocyclic groups include, but are not limited to, azetidine, chroman, dihydrofuran, dihydropyran, dioxane, dioxolane, hexahydroazepine, imidazolidine, imidazolidinone, imidazoline, imidazolinone, indoline, isochroman, isoindoline, isothiazoline, isothiazolidine, isoxazoline, isoxazolidine, morpholine, morpholinone, oxazoline, oxazolidine, oxazolidinone, oxetane, 2-oxohexahydroazepin, 2-oxopiperazine, 2-oxopiperidine, 2-oxopyrrolidine, piperazine, piperidine, pyran, pyrazolidine, pyrazoline, pyrrolidine, pyrroline, quinuclidine, tetrahydroquinoline, tetrahydroisoquinolines and oxindoles, tetrahydrofuran, tetrahydropyran, thiamorpholine, thiazoline, thiazolidine, thiomorpholine and N-oxides thereof.

The term “heteroaryl”, as used herein except where noted, represents a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, benzimidazole, benzisothiazole, benzisoxazole, benzofuran, benzothiazole, benzothiophene, benzotriazole, benzoxazole, carboline, cinnoline, furan, furazan, imidazole, indazole, indole, indolizine, isoquinoline, isothiazole, isoxazole, naphthyridine, oxadiazole, oxazole, phthalazine, pteridine, purine, pyran, pyrazine, pyrazole, pyridazine, pyridine, pyrimidine, pyrrole, quinazoline, quinoline, quinoxaline, tetrazole, thiadiazole, thiazole, thiophene, triazine, triazole, and N-oxides thereof.

Examples of heterocycloalkyls include azetidinyl, pyrrolidinyl, piperidinyl, piperazinyl, morpholinyl, tetrahydrofuranyl, imidazolinyl, pyrrolidin-2-one, piperidin-2-one, and thiomorpholinyl.

“Halogen” refers to fluorine, chlorine, bromine and iodine.

The term “mammal” “mammalian” or “mammals” includes humans, as well as animals, such as dogs, cats, horses, pigs and cattle.

Compounds described herein may contain one or more double bonds and may thus give rise to cis/trans isomers as well as other conformational isomers. The present invention includes all such possible isomers as well as mixtures of such isomers unless specifically stated otherwise.

The compounds of the present invention contain one or more asymmetric centers and may thus occur as racemates, racemic mixtures, single enantiomers, diastereomeric mixtures, and individual diastereomers.

It will be understood that, as used herein, references to the compounds of structural formula I are meant to also include the pharmaceutically acceptable salts, and also salts that are not pharmaceutically acceptable when they are used as precursors to the free compounds or in other synthetic manipulations.

The compounds of the present invention may be administered in the form of a pharmaceutically acceptable salt. The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

The pharmaceutical compositions of the present invention comprise compounds of the invention (or pharmaceutically acceptable salts thereof) as an active ingredient, a pharmaceutically acceptable carrier, and optionally one or more additional therapeutic agents or adjuvants. Such additional therapeutic agents can include, for example, i) opiate agonists or antagonists, ii) calcium channel antagonists, iii) 5HT receptor agonists or antagonists, iv) sodium channel antagonists, v) NMDA receptor agonists or antagonists, vi) COX-2 selective inhibitors, vii) NK1 antagonists, viii) non-steroidar anti-inflammatory drugs (“NSAID”), ix) selective serotonin reuptake inhibitors (“SSRI”) and/or selective serotonin and norepinephrine reuptake inhibitors (“SSNRI”), x) tricyclic antidepressant drugs, xi) norepinephrine modulators, xii) lithium, xiii) valproate, xiv) neurontin (gabapentin), and xv) sodium channel blockers. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The present compounds and compositions are useful for the treatment of chronic, visceral, inflammatory and neuropathic pain syndromes. They are useful for the treatment of pain resulting from traumatic nerve injury, nerve compression or entrapment, postherpetic neuralgia, trigeminal neuralgia, and diabetic neuropathy. The present compounds and compositions are also useful for the treatment of chronic lower back pain, phantom limb pain, chronic pelvic pain, neuroma pain, complex regional pain syndrome, chronic arthritic pain and related neuralgias, and pain associated with cancer, chemotherapy, HIV and HIV treatment-induced neuropathy. Compounds of this invention may also be utilized as local anesthetics. Compounds of this invention are useful for the treatment of irritable bowel syndrome and related disorders, as well as Crohn's disease.

The instant compounds have clinical uses for the treatment of epilepsy and partial and generalized tonic seizures. They are also useful for neuroprotection under ischaemic conditions caused by stroke or neural trauma and for treating multiple sclerosis. The present compounds are useful for the treatment of tachy-arrhythmias. Additionally, the instant compounds are useful for the treatment of neuropsychiatric disorders, including mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders.

In addition to primates, such as humans, a variety of other mammals can be treated according to the method of the present invention. For instance, mammals including, but not limited to, cows, sheep, goats, horses, dogs, cats guinea pigs, or other bovine, ovine, equine, canine, feline, rodent such as mouse, species can be treated. However, the method can also be practiced in other species, such as avian species (e.g., chickens).

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), α-adrenoreceptor antagonists, atypical anti-depressants, benzodiazepines, 5-HT_{1A} agonists or antagonists, especially 5-HT_{1A} partial agonists, neurokinin-1 receptor antagonists, corticotropin releasing factor (CRF) antagonists, and pharmaceutically acceptable salts thereof.

Further, it is understood that compounds of this invention can be administered at prophylactically effective dosage levels to prevent the above-recited conditions and disorders, as well as to prevent other conditions and disorders associated with sodium channel activity.

Creams, ointments, jellies, solutions, or suspensions containing the instant compounds can be employed for topical use. Mouth washes and gargles are included within the scope of topical use for the purposes of this invention.

Dosage levels from about 0.01 mg/kg to about 140 mg/kg of body weight per day are useful in the treatment of inflammatory and neuropathic pain, or alternatively about 0.5 mg to about 7 g per patient per day. For example, inflammatory pain may be effectively treated by the administration of from about 0.01 mg to about 75 mg of the compound per kilogram of body weight per day, or alternatively about 0.5 mg to about 3.5 g per patient per day. Neuropathic pain may be effectively treated by the administration of from about 0.01 mg to about 125 mg of the compound per kilogram of body weight per day, or alternatively about 0.5 mg to about 5.5 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may ary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors. Such patient-related factors include the age, body weight, general health, sex, and diet of the patient. Other factors include the time and route of administration, rate of excretion, drug combination, and the severity of the particular disease undergoing therapy.

In practice, the compounds of the invention, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I, Ia, Ib, Id or Ie. The compounds of the invention, or pharmaceutically acceptable salts thereof, can also be included in pharmaceutical compositions in combination with one or more therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, and disintegrating agents can be used to form oral solid preparations such as powders, capsules and tablets. Because of their ease of administration, tablets and capsules are advantageous oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet advantageously contains from about 0.1 mg to about 500 mg of the active ingredient and each cachet or capsule advantageously containing from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and thus should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, and dusting powder. Further, the compositions can be in a form suitable for use in transdermal devices. These formulations may be prepared, utilizing a compound represented of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid, such as, for example, where the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, and preservatives (including anti-oxidants). Furthermore, other adjuvants can be included to render the formulation isotonic with the blood of the intended recipient. Compositions containing a compound of the invention, or pharmaceutically acceptable salts thereof, can also be prepared in powder or liquid concentrate form.

The compounds and pharmaceutical compositions of this invention have been found to block sodium channels. Accordingly, an aspect of the invention is the treatment and prevention in mammals of conditions that are amenable to amelioration through blockage of neuronal sodium channels by administering an effective amount of a compound of this invention. Such conditions include, for example, acute pain, chronic pain, visceral pain, inflammatory pain and neuropathic pain. The instant compounds and compositions are useful for treating and preventing the above-recited conditions, including acute pain, chronic pain, visceral pain, inflammatory pain and neuropathic pain, in humans and non-human mammals such as dogs and cats. It is understood that the treatment of mammals other than humans refers to the treatment of clinical conditions in non-human mammals that correlate to the above-recited conditions.

Further, as described above, the instant compounds can be utilized in combination with one or more therapeutically active compounds. In particular, the inventive compounds can be advantageously used in combination with i) opiate agonists or antagonists, ii) calcium channel antagonists, iii) 5HT receptor agonists or antagonists, including 5-HT_{1A} agonists or antagonists, and 5-HT_{1A} partial agonists, iv) sodium channel antagonists, v) N-methyl-D-aspartate (NMDA) receptor agonists or antagonists, vi) COX-2 selective inhibitors, vii) neurokinin receptor 1 (NK1) antagonists, viii) non-steroidal anti-inflammatory drugs (NSAID), ix) selective serotonin reuptake inhibitors (SSRI) and/or selective serotonin and norepinephrine reuptake inhibitors (SSNRI), x) tricyclic antidepressant drugs, xi) norepinephrine modulators, xii) lithium, xiii) valproate, xiv) norepinephrine reuptake inhibitors, xv) monoamine oxidase inhibitors (MAOIs), xvi) reversible inhibitors of monoamine oxidase (RIMAs), xvii) □-adrenoreceptor antagonists, xviii) atypical anti-depressants, xix) benzodiazepines, xx) corticotropin releasing factor (CRF) antagonists, and xxi) neurontin (gabapentin).

The abbreviations used herein have the following meanings (abbreviations not shown here have their meanings as commonly used unless specifically stated otherwise): Ac (acetyl), Bn (benzyl), Boc (tertiary-butoxy carbonyl), CAMP (cyclic adenosine-3′,5′-monophosphate), DAST ((diethylamino)sulfur trifluoride), DBU (1,8-diazabicyclo[5.4.0]undec-7-ene), DIBAL (diisobutylaluminum hydride), DMAP (4-(dimethylamino)pyridine), DMF (N,N-dimethylformamide), EDC (1-(3-dimethylaminopropyl)-3-ethylcarbodiimide hydrochloride), Et₃N (triethylamine), GST (glutathione transferase), HOBt (1-hydroxybenzotriazole), LAH (lithium aluminum hydride), Ms (methanesulfonyl; mesyl; or SO₂Me), MsO (methanesulfonate or mesylate), NBS (N-bromosuccinimide), NCS(N-chlorosuccinimide), NSAID (non-steroidal anti-inflammatory drug), PDE (Phosphodiesterase), Ph (Phenyl), r.t. or RT (room temperature), Rac (Racemic), SAM (aminosulfonyl; sulfonamide or SO₂NH₂), SPA (scintillation proximity assay), Th (2- or 3-thienyl), TFA (trifluoroacetic acid), THF (Tetrahydrofuran), Thi (Thiophenediyl), TLC (thin layer chromatography), TMEDA (N,N,N′,N′-tetramethylethylenediamine), TMSI (trimethylsilyl iodide), Th or trityl (N-triphenylmethyl), C₃H₅ (Allyl), Me (methyl), Et (ethyl), n-Pr (normal propyl), i-Pr (isopropyl), n-Bu (normal butyl), i-Butyl (isobutyl), s-Bu (secondary butyl), t-Bu (tertiary butyl), c-Pr (cyclopropyl), c-Bu (cyclobutyl), c-Pen (cyclopentyl), c-Hex (cyclohexyl).

The present compounds can be prepared according to the general Schemes provided below as well as the procedures provided in the Examples. The following Schemes and Examples further describe, but do not limit, the scope of the invention.

Unless specifically stated otherwise, the experimental procedures were performed under the following conditions: All operations were carried out at room or ambient temperature; that is, at a temperature in the range of 18-25° C. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) or by high-pressure liquid chromatography-mass spectrometry (HPLC-MS), and reaction times are given for illustration only. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. Broad; etc. In addition, “Ar” signifies an aromatic signal. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### ASSAY EXAMPLE 1

### Fluorescent Assay for Cav2.2 Channels Using Potassium Depolarization to Initiate Channel Opening

Human Cav2.2 channels were stably expressed in KEK293 cells along with alpha2-delta and beta subunits of voltage-gated calcium channels. An inwardly rectifying potassium channel (Kir2.3) was also expressed in these cells to allow more precise control of the cell membrane potential by extracellular potassium concentration. At low bath potassium concentration, the membrane potential is relatively negative, and is depolarized as the bath potassium concentration is raised. In this way, the bath potassium concentration can be used to regulate the voltage-dependent conformations of the channels. Compounds are incubated with cells in the presence of low (4 mM) potassium or elevated (12, 25 or 30 mM) potassium to determine the affinity for compound block of resting (closed) channels at 4 mM potassium or affinity for block of open and inactivated channels at 12, 25 or 30 mM potassium. After the incubation period, Cav2.2 channel opening is triggered by addition of higher concentration of potassium (70 mM final concentration) to further depolarize the cell. The degree of state-dependent block can be estimated from the inhibitory potency of compounds after incubation in different potassium concentrations.

Calcium influx through Cav2.2 channels is determined using a calcium-sensitive fluorescent dye in combination with a fluorescent plate reader. Fluorescent changes were measured with either a VIPR (Aurora Instruments) or FLIPR (Molecular Devices) plate reader.

### Protocol

1. Seed cells in Poly-D-Lysine Coated 96- or 384-well plate and keep in a 37° C.-10% CO₂ incubator overnight
2. Remove media¹, wash cells with 0.2 ml (96-well plate) or 0.05 ml (384-well plate) Dulbecco's Phosphate Buffered Saline (D-PBS) with calcium & magnesium (Invitrogen; 14040)
3. Add 0.1 ml (96-well plate) or 0.05 ml (384-well plate) of 4 μM fluo-4 (Molecular Probes; F-14202) and 0.02% Pluronic acid (Molecular Probes; P-3000) prepared in D-PBS with calcium & magnesium (Invitrogen; 14040) supplemented with 10 mM Glucose & 10 mM Hepes/NaOH; pH 7.4
4. Incubate in the dark at 25° C. for 60-70 min
5. Remove dye², wash cells with 0.1 ml (96-well plate) or 0.06 ml (384-well plate) of 4, 12, 25, or 30 mM Potassium Pre-polarization Buffer. (PPB)
6. Add 0.1 ml (96-well plate) or 0.03 ml (384-well plate) of 4, 12, 25, 30 mM PPB, with or without test compound
7. Incubate in the dark at 25° C. for 30 min
8. Read cell plate on VIPR instrument, Excitation=480 nm, Emission=535 nm
9. With VIPR continuously reading, add 0.1 ml (96-well plate) or 0.03 ml (384-well plate) of Depolarization Buffer, which is 2× the final assay concentration, to the cell plate.

| | | | | 140 mM K |
|---|---|---|---|---|
| | | | | Depolarizing |
| 4 mM PPB | 12 mM PPB | 25 mM PPB | 30 mM PPB | Buffer |
| 146 mM | 138 mM | 125 mM | 120 mM | 10 |
| NaCl | NaCl | NaCl | NaCl | NaCl |
| 4 mM KCl | 12 mM KCl | 25 mM KCl | 30 mM KCl | 140 KCl |
| 0.8 mM | 0.8 mM | 0.8 mM | 0.8 mM | 0.8 mM |
| CaCl₂ | CaCl₂ | CaCl₂ | CaCl₂ | CaCl₂ |
| 1.7 MgCl₂ | 1.7 MgCl₂ | 1.7 MgCl₂ | 1.7 MgCl₂ | 1.7 MgCl₂ |
| 10 HEPES | 10 HEPES | 10 HEPES | 10 HEPES | 10 HEPES |
| pH = 7.2 | pH = 7.2 | pH = 7.2 | pH = 7.2 | pH = 7.2 |

### ASSAY EXAMPLE 2

### Electrophysiological Measurement of Block of Cav2.2 Channels Using Automated Electrophysiology Instruments

Block of N-type calcium channels is evaluated utilizing the IonWorks HT 384 well automated patch clamp electrophysiology device. This instrument allows synchronous recording from 384 wells (48 at a time). A single whole cell recording is made in each well. Whole cell recording is established by perfusion of the internal compartment with amphotericin B.

The voltage protocol is designed to detect use-dependent block. A 2 Hz train of depolarizations (twenty 25 ms steps to +20 mV). The experimental sequence consists of a control train (pre-compound), incubation of cells with compound for 5 minutes, followed by a second train (post-compound). Use dependent block by compounds is estimated by comparing fractional block of the first pulse in the train to block of the 20th pulse.

### Protocol

Parallel patch clamp electrophysiology is performed using IonWorks HT (Molecular Devices Corp.) essentially as described by Kiss and colleagues [Kiss et al. 2003; Assay and Drug Development Technologies, 1: 127-135]. Briefly, a stable HEK 293 cell line (referred to as CBK) expressing the N-type calcium channel subunits (alpha_{1B}, alpha₂-delta, beta₃ₐ,) and an inwardly rectifying potassium channel (Kᵢᵣ2.3) is used to record barium current through the N-type calcium channel. Cells are grown in T75 culture plates to 60-90% confluence before use. Cells are rinsed 3× with 10 ml PBS (Ca/Mg-free) followed by addition of 1.0 ml 1× trypsin to the flask. Cells are incubated at 37° C. until rounded and free from plate (usually 1-3 min). Cells are then transferred to a 15 ml conical tube with 13 ml of CBK media containing serum and antibiotics and spun at setting 2 on a table top centrifuge for 2 min. The supernatant is poured off and the pellet of cells is resuspended in external solution (in mM): 120 NaCl, 20 BaCl₂, 4.5 KCl, 0.5 MgCl₂, 10 HEPES, 10 Glucose, pH=7.4). The concentration of cells in suspension is adjusted to achieve 1000-3000 cells per well. Cells are used immediately once they have been resuspended. The internal solution is (in mM): 100 K-Gluconate, 40 KCl, 3.2 MgCl₂, 3 EGTA, 5 HEPES, pH 7.3 with KOH. Perforated patch whole cell recording is achieved by added the perforating agent amphotericin B to the internal solution. A 36 mg/ml stock of amphtericn B is made fresh in DMSO for each run. 166 □l of this stock is added to 50 ml of internal solution yielding a final working solution of 120 ug/ml.

Voltage protocols and the recording of membrane currents are performed using the IonWorks HT software/hardware system. Currents are sampled at 1.25 kHz and leakage subtraction is performed using a 10 mV step from the holding potential and assuming a linear leak conductance. No correction for liquid junction potentials is employed. Cells are voltage clamped at −70 mV for 10 s followed by a 20 pulse train of 25 ms steps to +20 mV at 2 Hz. After a control train, the cells are incubated with compound for 5 minutes and a second train is applied. Use dependent block by compounds is estimated by comparing fractional block of the first pulse to block of the 20th pulse. Wells with seal resistances less than 70 MOhms or less than 0.1 nA of Ba current at the test potential (+20 mV) are excluded from analysis. Current amplitudes are calculated with the IonWorks software. Relative current, percent inhibition and IC50s are calculated with a custom Excel/Sigmaplot macro.

Compounds are added to cells with a fluidics head from a 96-well compound plate. To compensate for the dilution of compound during addition, the compound plate concentration is 3× higher than the final concentration on the patch plate.

Two types of experiments are generally performed: screens and titrations. In the screening mode, 10-20 compounds are evaluated at a single concentration (usually 3 uM). The percent inhibition is calculated from the ratio of the current amplitude in the presence and absence of compound, normalized to the ratio in vehicle control wells. For generation of IC50s, a 10-point titration is performed on 2-4 compounds per patch plate. The range of concentrations tested is generally 0.001 to 20 uM. IC50s are calculated from the fits of the Hill equation to the data. The form of the Hill equation used is: Relative Current=Max−Min)/(1+(conc/IC50)̂slope))+Min. Vehicle controls (DMSO) and 0.3 mM CdCl₂ (which inhibits the channel completely) are run on each plate for normalization purposes and to define the Max and Min.

### ASSAY EXAMPLE 3

### Electrophysiological Measurement of Block of Cav2.2 Channels Using Whole Cell Voltage Clamp and Using PatchXpress Automated Electrophysiology Instrument

Block of N-type calcium channels is evaluated utilizing manual and automated (PatchXpress) patch clamp electrophysiology. Voltage protocols are designed to detect state-dependent block. Pulses (50 ms) are applied at a slow frequency (0.067 Hz) from polarized (−90 mV) or depolarized (−40 mV) holding potentials. Compounds which preferentially block inactivated/open channels over resting channels will have higher potency at −40 mV compared to −90 mV.

### Protocol:

A stable HEK 293 cell line (referred to as CBK) expressing the N-type calcium channel subunits (alpha_{1B}, alpha₂-delta, beta₃ₐ,) and an inwardly rectifying potassium channel (Kᵢᵣ2.3) is used to record barium current through the N-type calcium channel. Cells are grown either on poly-D-lysine coated coverglass (manual EP) or in T75 culture plates (PatchXpress). For the PatchXpress, cells are released from the flask using tryspin. In both cases, the external solution is (in mM): 120 NaCl, 20 BaCl₂, 4.5 KCl, 0.5 MgCl₂, 10 HEPES, 10 Glucose, pH 7.4 with NaOH. The internal solution is (in mM): 130 CsCl, 10 EGTA, 10 HEPES, 2 MgCl₂, 3 MgATP, pH 7.3 with CsOH.

Barium currents are measured by manual whole-cell patch clamp using standard techniques (Hamill et. al. Pfluegers Archiv 391:85-100 (1981)). Microelectrodes are fabricated from borosilicate glass and fire-polished. Electrode resistances are generally 2 to 4 MOhm when filled with the standard internal saline. The reference electrode is a silver-silver chloride pellet. Voltages are not corrected for the liquid junction potential between the internal and external solutions and leak is subtracted using the P/n procedure. Solutions are applied to cells by bath perfusion via gravity. The experimental chamber volume is ˜0.2 ml and the perfusion rate is 0.5-2 ml/min. Flow of solution through the chamber is maintained at all times. Measurement of current amplitudes is performed with PULSEFIT software (HEKA Elektronik).

PatchXpress (Molecular Devices) is a 16-well whole-cell automated patch clamp device that operates asynchronously with fully integrated fluidics. High resistance (gigaohm) seals are achieved with 50-80% success. Capacitance and series resistance compensation is automated. No correction for liquid junction potentials is employed. Leak is subtracted using the P/n procedure. Compounds are added to cells with a pipettor from a 96-well compound plate. Voltage protocols and the recording of membrane currents are performed using the PatchXpress software/hardware system. Current amplitudes are calculated with DataXpress software.

In both manual and automated patch clamp, cells are voltage clamped at 40 mV or −90 mV and 50 ms pulses to +20 mV are applied every 15 sec (0.067 Hz). Compounds are added in escalating doses to measure % Inhibition. Percent inhibition is calculated from the ratio of the current amplitude in the presence and absence of compound. When multiple doses are achieved per cell, IC50s are calculated. The range of concentrations tested is generally 0.1 to 30 uM. IC50s are calculated from the fits of the Hill equation to the data. The form of the Hill equation used is: Relative Current=1/(1+(conc/IC50)̂slope)).

### In Vivo Assay: (Rodent CFA Model):

Male Sprague Dawley rats (300-400 gm) were administered 200 microl CFA (Complete Freund's Adjuvant) three days prior to the study. CFA is mycobacterium tuberculosis suspended in saline (1:1; Sigma) to form an emulsion that contains 0.5 mg mycobacterium/ml. The CFA was injected into the plantar area of the left hind paw.

Rats are fasted the night before the study only for oral administration of compounds. On the morning of test day using a Ugo Basile apparatus, 2 baseline samples are taken 1 hour apart. The rat is wrapped in a towel. Its paw is placed over a ball bearing and under the pressure device. A foot pedal is depressed to apply constant linear pressure. Pressure is stopped when the rat withdraws its paw, vocalizes, or struggles. The right paw is then tested. Rats are then dosed with compound and tested at predetermined time points. Compounds were prepared in DMSO(15%)/PEG300(60%)/Water(25%) and were dosed in a volume of 2 ml/kg.

Percent maximal possible effect (% MPE) was calculated as: (post-treatment−pre-treatment)/(pre-injury threshold−pre-treatment)×100. The % responder is the number of rats that have a MPE.30% at any time following compound administration. The effect of treatment was determined by one-way ANOVA Repeated Measures Friedman Test with a Dunn's post test.

### Methods of Synthesis

Compounds of the present invention can be prepared according to the Schemes provided below as well as the procedures provided in the Examples. The substituents are the same as in the above Formulas except where defined otherwise or otherwise apparent to the ordinary skilled artisan.

The novel compounds of the present invention can be readily synthesized using techniques known to those skilled in the art, such as those described, for example, in Advanced Organic Chemistry, March, 5^{th} Ed., John Wiley and Sons, New York, N.Y., 2001, Advanced Organic Chemistry, Carey and Sundberg, Vol. A and B, 3^{rd} Ed., Plenum Press, Inc., New York, N.Y., 1990; Protective groups in Organic Synthesis, Green and Wuts, 2^{nd} Ed., John Wiley and Sons, New York, N.Y., 1991; Comprehensive Organic Transformations, Larock, VCH Publishers, Inc., New York, N.Y., 1988; Handbook of Heterocyclic Chemistry, Katritzky and Pozharskii, 2^{nd} Ed., Pergamon, New York, N.Y., 2000 and references cited therein. The starting materials for the present compounds may be prepared using standard synthetic transformations of chemical precursors that are readily available from commercial sources, including Aldrich Chemical Co. (Milwaukee, Wis.); Sigma Chemical Co. (St. Louis, Mo.); Lancaster Synthesis (Windham, N.H.); Ryan Scientific (Columbia, S.C.); Maybridge (Cornwall, UK); Matrix Scientific (Columbia, S.C.); Arcos, (Pittsburgh, Pa.) and Trans World Chemicals (Rockville, Md.).

The procedures described herein for synthesizing the compounds may include one or more steps of protecting group manipulations and of purification, such as, recrystallization, distillation, column chromatography, flash chromatography, thin-layer chromatography (TLC), radial chromatography and high-pressure chromatography (HPLC). The products can be characterized using various techniques well known in the chemical arts, including proton and carbon-13 nuclear magnetic resonance (¹H and ¹³C NMR), infrared and ultraviolet spectroscopy (IR and UV), X-ray crystallography, elemental analysis and HPLC and mass spectrometry (HPLC-MS). Methods of protecting group manipulation, purification, structure identification and quantification are well known to one skilled in the art of chemical synthesis.

Appropriate solvents are those which will at least partially dissolve one or all of the reactants and will not adversely interact with either the reactants or the product. Suitable solvents are aromatic hydrocarbons (e.g, toluene, xylenes), halogenated solvents (e.g, methylene chloride, chloroform, carbontetrachloride, chlorobenzenes), ethers (e.g. diethyl ether, diisopropylether, tert-butyl methyl ether, diglyme, tetrahydrofuran, dioxane, anisole), nitriles (e.g, acetonitrile, propionitrile), ketones (e.g, 2-butanone, dithyl ketone, tert-butyl methyl ketone), alcohols (e.g, methanol, ethanol, n-propanol, iso-propanol, n-butanol, t-butanol), N,N-dimethyl formamide (DMF), dimethylsulfoxide (DMSO) and water. Mixtures of two or more solvents can also be used. Suitable bases are, generally, alkali metal hydroxides, alkaline earth metal hydroxides such as lithium hydroxide, sodium hydroxide, potassium hydroxide, barium hydroxide, and calcium hydroxide; alkali metal hydrides and alkaline earth metal hydrides such as lithium hydride, sodium hydride, potassium hydride and calcium hydride; alkali metal amides such as lithium amide, sodium amide and potassium amide; alkali metal carbonates and alkaline earth metal carbonates such as lithium carbonate, sodium carbonate, cesium carbonate, sodium hydrogen carbonate, and cesium hydrogen carbonate; alkali metal alkoxides and alkaline earth metal alkoxides such as sodium methoxide, sodium ethoxide, potassium tert-butoxide and magnesium ethoxide; alkali metal alkyls such as methyllithium, n-butyllithium, sec-butyllithium, t-bultyllithium, phenyllithium, alkyl magnaesium halides, organic bases such as trimethylamine, triethylanine, triisopropylamine, N,N-diisopropylethylamine, piperidine, N-methyl piperidine, morpholine, N-methyl morpholine, pyridine, collidines, lutidines, and 4-dimethylaminopyridine; and bicyclic amines such as DBU and DABCO.

As described previously, in preparing the compositions for oral dosage form, any of the usual pharmaceutical media can be employed. For example, in the case of oral liquid preparations such as suspensions, elixirs and solutions, water, glycols, oils, alcohols, flavoring agents, preservatives, coloring agents and the like may be used; or in the case of oral solid preparations such as powders, capsules and tablets, carriers such as starches, sugars, microcrystalline cellulose, diluents, granulating agents, lubricants, binders, disintegrating agents, and the like may be included. Because of their ease of administration, tablets and capsules represent the most advantageous oral dosage unit form in which solid pharmaceutical carriers are employed. If desired, tablets may be coated by standard aqueous or nonaqueous techniques. In addition to the common dosage forms set out above, controlled release means and/or delivery devices may also be used in administering the instant compounds and compositions.

It is understood that the functional groups present in compounds described in the Schemes below can be further manipulated, when appropriate, using the standard functional group transformation techniques available to those skilled in the art, to provide desired compounds described in this invention.

It is also understood that compounds listed in the Schemes and Tables below that contain one or more stereocenters may be prepared as single enantiomers or diastereomers, or as mixtures containing two or more enantiomers or diastereomers in any proportion.

Other variations or modifications, which will be obvious to those skilled in the art, are within the scope and teachings of this invention. This invention is not to be limited except as set forth in the following claims.

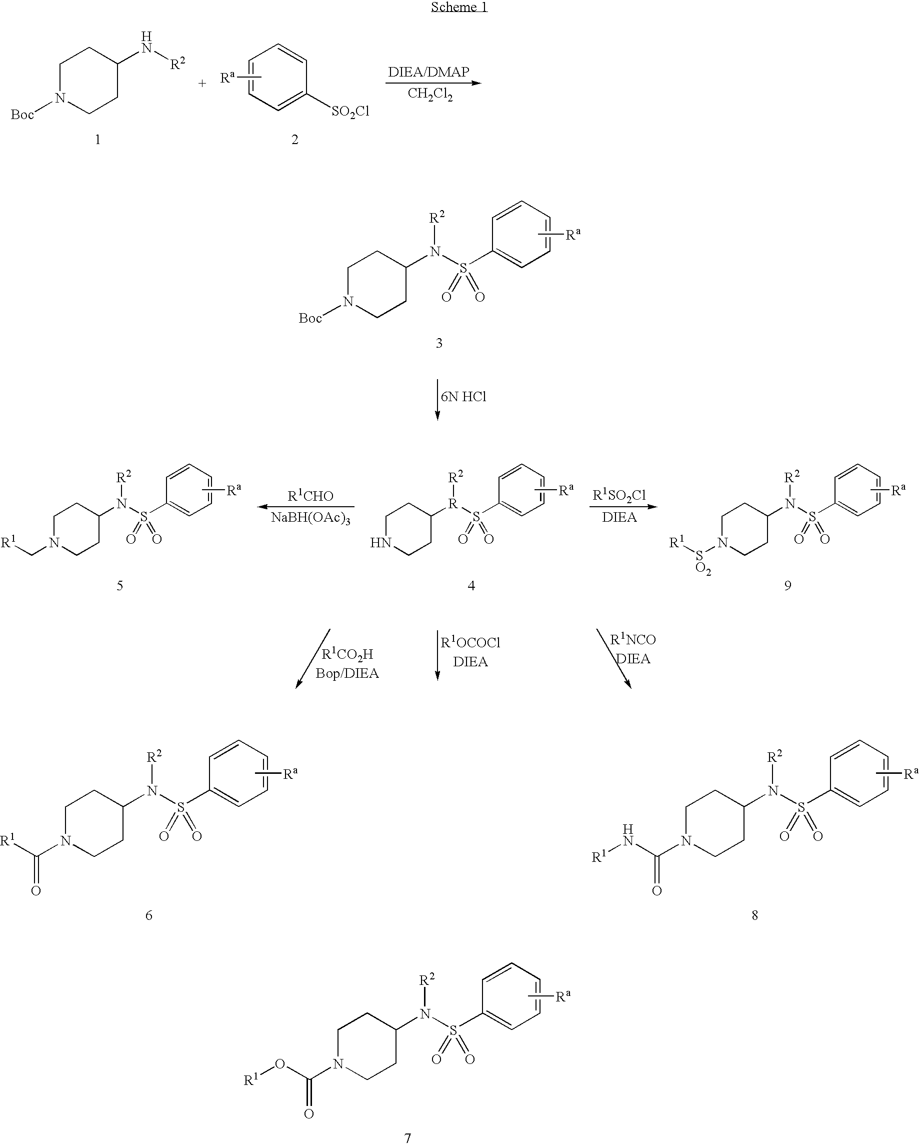

The N-protected 4-amino piperidine 1 can be reacted with an appropriate substituted arylsulfonyl chloride 2 to give the sulfonamide 3. Removal of N-protecting group can provide a versatile intermediate 4, which can be then reacted with appropriate reagents, as shown in Scheme 1, to provide corresponding amines 5, amides 6, carbamates 7, ureas 8 or sulfonamides 9.

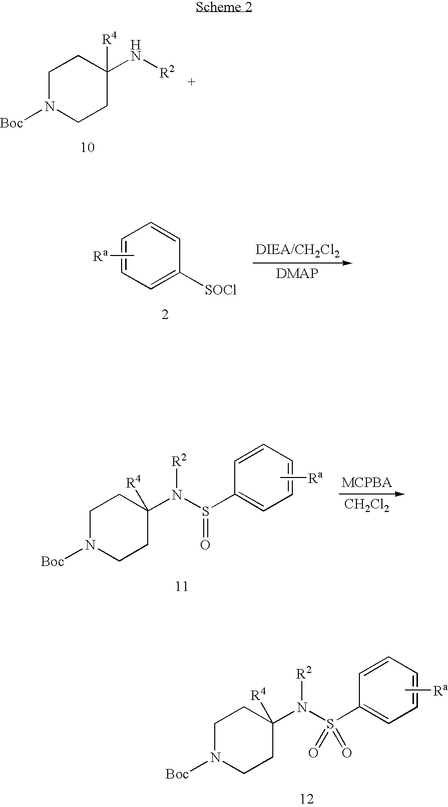

In case of a sterically hindered amine 10, the amine can be reacted with an appropriate sulfinyl chloride 2 to give sulfinamide 11 which can be further oxidized with metachloroperbenzoic acid to provide the sulfonamide 12, as illustrated in Scheme 2.

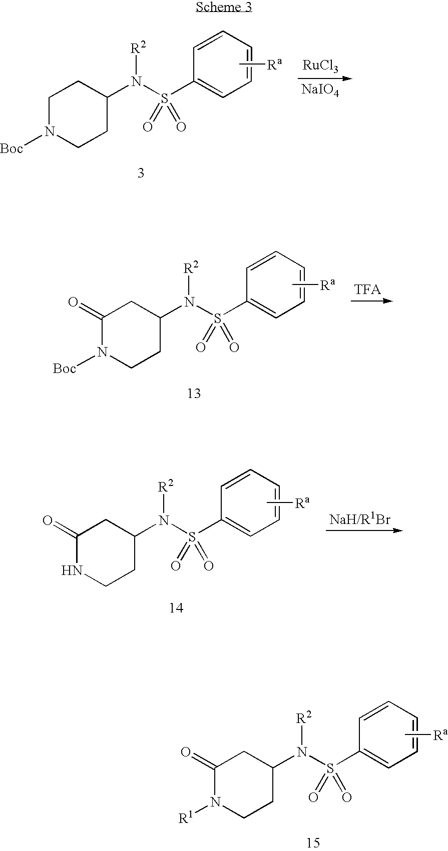

The N-protected piperidine 3 can be oxidized with NaIO₄ using RuCl₃ as the catalyst to provide the corresponding piperidone 13 [Chem. Pharm. Bull., 36: 3125 (1988)], which can be deprotected and subjected to N-alkylation under appropriate conditions to give the corresponding N-alkylated product 15 as illustrated in Scheme 3.

### EXAMPLE 1

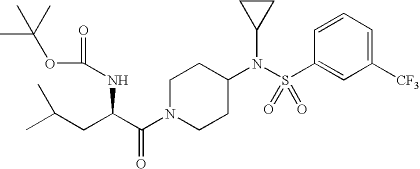

### Step 1: Preparation of

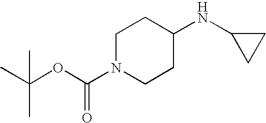

A solution of tert-butyl 4-oxopiperidine-1-carboxylate (1 g) and cyclopropylamine (0.43 g) in methanol (10 mL) was stirred at 45° C. for two hours. The reaction was cooled to room temperature, and NaBH₄ (0.17 g) was added in batches to the reaction. After stirring at room temperature for 15 min, the reaction mixture was concentrated in vacuo, and the residue was partitioned between 5% KOH solution and ethyl acetate. The organics phase was washed with water and dried over Na₂SO₄, filtered and concentrated to give 1.06 g of the crude amine product as colorless oil. ¹H NMR (CDCl₃): 4.05 (m, 2H), 2.8 (b, 2H), 2.76 (m, 1H), 2.17 (m, 1H), 1.95 (m, 2H), 1.46 (s, 9H), 1.23 (m, 2H), 0.5 (m, 2H), 0.35 (m, 2H).

### Step 2: Preparation of

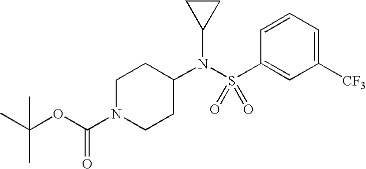

To a solution of the amine (0.5 g) (from step 1 above) in CH₂Cl₂ (15 mL) were added diisopropylethylamine (1.1 mL), 2 grains of N,N-dimethylaminopyridine and 3-(trifluoromethyl)benzenesulfonyl chloride (0.56 g) at 0° C. The reaction mixture was stirred at room temperature overnight. The reaction mixture was washed with water and concentrated in vacuo. The crude product obtained was purified by silica gel chromatography using ethyl acetate/hexane (1:9 to 1:6) to afford 1.05 g the titled product as a colorless oil. ¹H NMR (CDCl₃): 8.17 (d, J=7.7 Hz, 1H), 8.14 (s, 1H), 8.0 (d, J=8.0 Hz, 1H), 7.85 (t, J=7.8 Hz, 1H), 4.1 (m, 2H), 4.05 (m, 1H), 2.78 (b, 2H), 2.03 (m, 1H), 1.8 (m, 2H), 1.54 (d, J=13 Hz, 1H), 1.44 (s, 9H), 0.92 (m, 2H), 0.8 (m, 2H). MS: m/e 449 (M+1)⁺, 393 (M−^{t}Bu)⁺.

### Step 3: Preparation of

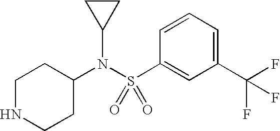

The Boc protected piperidine compound (Step 2, 350 mg) was reacted with 6N HCl (5 mL) and ethyl acetate (5 mL) for three hours at room temperature. The reaction mixture was made alkaline with the addition of 10% KOH, and then extracted with ethyl acetate/ether (1:1). The organic phase was washed with water and dried over Na₂SO₄, filtered and concentrated to give 220 mg of the titled amine compound as a white solid. ¹H NMR (CDCl₃): 8.17 (s, 1H), 8.09 (d, J=7.8 Hz, 1H), 7.87 (d, J=7.8 Hz, 1H), 7.71 (t, J=7.9 Hz, 1H), 3.97 (m, 1H), 3.12 (m, 2H), 2.65 (td, J=8 Hz, J=2.1 Hz, 2H), 2.03 (m, 1H), 1.88 (m, 2H), 1.58 (m, 2H), 1.02 (m, 2H), 0.8 (m, 2H). MS: m/e 349 (M+1)⁺.

### Step 4: Preparation of

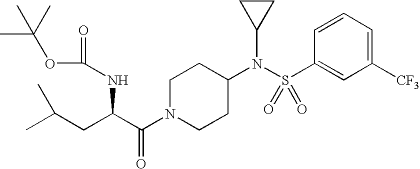

The amine (Step 3, 600 mg) was dissolved in dimethyl formamide (10 mL), and to the solution was added BOP regagent (benzotriazole-1-yl-oxy-tris-(dimethylamino)-phosphonium hexafluorophosphate) (827 mg), (R)—N-Boc-valine (361 mg), and diisopropylethylamine (1.1 mL). After stirring 18 h at 40° C., the reaction was diluted with ether (75 mL) and washed with saturated aqueous NH₄Cl (75 mL). The organic layer was washed with saturated aqueous NaHCO₃, dried (Na₂SO₄), and concentrated. The crude product was purified by silica gel chromatography with a gradient of ethyl acetate/hexanes (1:3 to 2:3) affording the title compound as a white solid. ¹H NMR (CDCl₃): 8.16 (s, 1H), 8.09 (d, J=8.0 Hz, 1H), 7.89 (d, J=7.0 Hz, 1H), 7.72 (t, J=8.0 Hz, 1H), 5.23 (m, 1H), 4.66 (m, 2H), 4.02 (m, 2H), 3.07 (m, 1H), 2.56 (m, 1H), 1.97 (m, 2H), 1.84 (m, 2H), 1.71 (m, 4H), 1.45 (s, 9H), 1.39 (m, 2H), 1.00 (t, J=7 Hz, 3H), 0.94 (t, J=7 Hz, 3H), 0.79 (m, 4H). MS: m/e 584 (M+1+Na)⁺.

TABLE 1

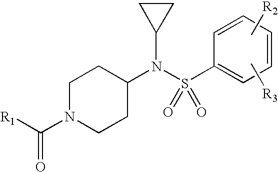

| | | | | Mass |
|---|---|---|---|---|
| | | | | (M + H) |
| EXAMPLES | R₁ | R₂ | R₃ | m/e |
| 2 | HOOCCH₂CH₂— | 4-Cl | H | 415 |
| 3 | Ph-NH— | 3-CF₃ | H | 468 |
| 4 | 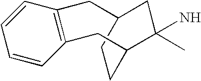 | 3-CF₃ | H | 562 |
| 5 | 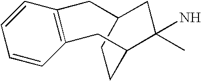 | H | H | 494 |
| 6 | 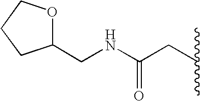 | 3-CF₃ | H | 532 |
| 7 | 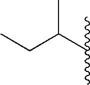 | 3-CF₃ | H | 433 |
| 8 | 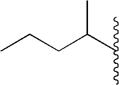 | 3-CF₃ | H | 447 |
| 9 | 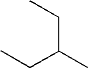 | 3-CF₃ | H | 407 |
| 10 | 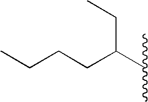 | 3-CF₃ | H | 475 |
| 11 | (CH₃)₃C— | 3-CF₃ | H | 433 |
| 12 | 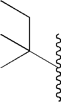 | 3-CF₃ | H | 447 |
| 13 | 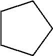 | 3-CF₃ | H | 445 |
| 14 | 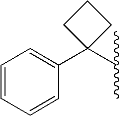 | 3-CF₃ | H | 507 |
| 15 | 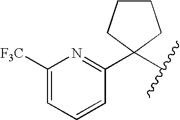 | 3-CF₃ | H | 590 |
| 16 | (CH₃)₃C—O— | 3-CF₃ | 5-CF₃ | 517 |
| 17 | 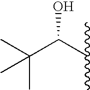 | 3-CF₃ | H | 463 |
| 18 | 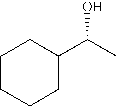 | 3-CF₃ | H | 489 |
| 19 | 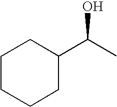 | 3-CF₃ | H | 489 |
| 20 | 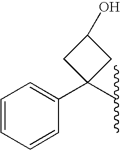 | 3-CF₃ | H | 523 |
| 21 | 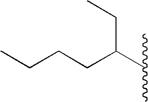 | 3-CF₃ | 5-CF₃ | 543 |
| 22 | 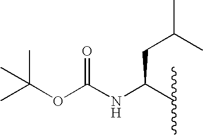 | 3-CF₃ | H | 561 |
| 23 | 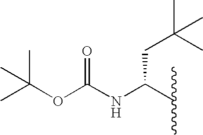 | 3-CF₃ | H | 576 |
| 24 | 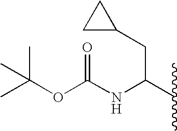 | 3-CF₃ | H | 560 |
| 25 | 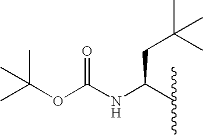 | 3-CF₃ | H | 576 |
| 26 | (CH₃)₃C—O— | 2-Br | H | 460 |
| 27 | (CH₃)₃C—O— | 2-OCH₃ | 3-Br | 490 |
| 28 | CH₃O—CH₂CH₂—O— | 3-CF₃ | H | 451 |
| 29 | CH₃CH₂CH₂CH₂CH₂—O— | 3-CF₃ | H | 463 |
| 30 | 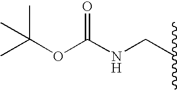 | 3-CF₃ | H | 506 |
| 31 | 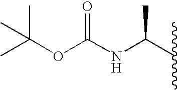 | 3-CF₃ | H | 520 |
| 32 | 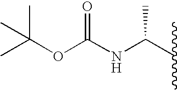 | 3-CF₃ | H | 520 |
| 33 | 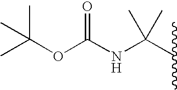 | 3-CF₃ | H | 533 |
| 34 | 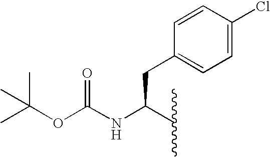 | 3-CF₃ | H | 631 |
| 35 | 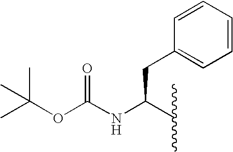 | 3-CF₃ | H | 596 |
| 36 | 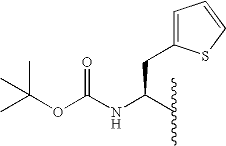 | 3-CF₃ | H | 616 |
| 37 | 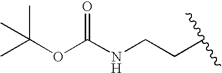 | 3-CF₃ | H | 520 |
| 38 | 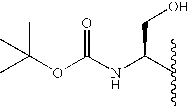 | 3-CF₃ | H | 536 |
| 39 | 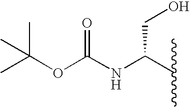 | 3-CF₃ | H | 536 |
| 40 | 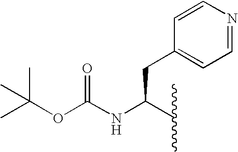 | 3-CF₃ | H | 611 |
| 41 | (CH₃)₃CH₂—O— | 3-CF₃ | H | 462 |
| 42 | (CH₃)₃—O— | 3-Cl | 5-Cl | 450 |
| 43 | H | 3-Cl | 5-Cl | 350 |
| 44 | H | 3-CF₃ | 5-CF₃ | 417 |
| 45 | 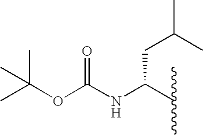 | 3-Br | H | 573 |
| 46 | 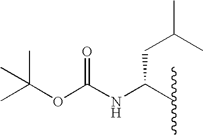 | 3-Cl | 5-Cl | 563 |
| 47 | 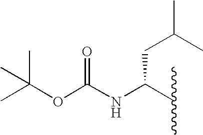 | 2-OCH₃ | 5-Br | 603 |
| 48 | 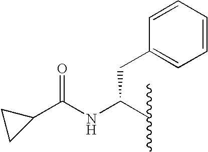 | 3-CF₃ | H | 563 |
| 49 | 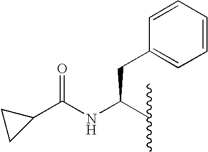 | 3-CF₃ | H | 563 |
| 50 | 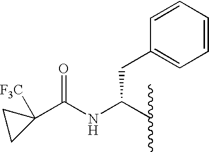 | 3-CF₃ | H | 632 |
| 51 | 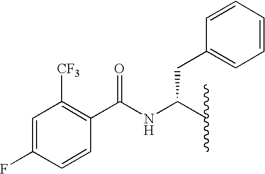 | 3-CF₃ | H | 685 |
| 52 | 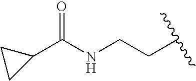 | 3-CF₃ | H | 487 |
| 53 | 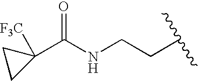 | 3-CF₃ | H | 556 |
| 54 | 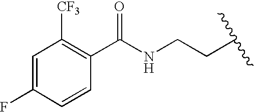 | 3-CF₃ | H | 610 |
| 55 | 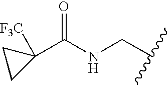 | 3-CF₃ | H | 542 |
| 56 | 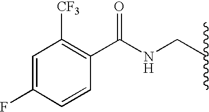 | 3-CF₃ | H | 596 |
| 57 | 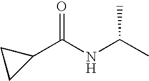 | 3-CF₃ | H | 488 |
| 58 | 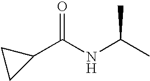 | 3-CF₃ | H | 488 |
| 59 | 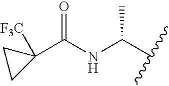 | 3-CF₃ | H | 556 |
| 60 | 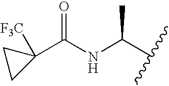 | 3-CF₃ | H | 556 |
| 61 | 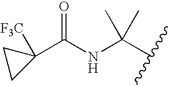 | 3-CF₃ | H | 570 |
| 62 | 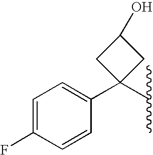 | 3-CF₃ | H | 540 |
| 63 | 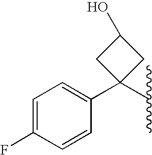 | 3-CF₃ | H | 540 |
| 64 | 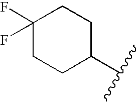 | 3-CF₃ | H | 495 |
| 65 | 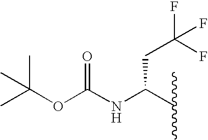 | 3-CF₃ | H | 588 |
| 66 | 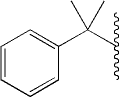 | 3-CF₃ | H | 495 |
| 67 | 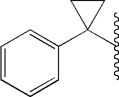 | 3-CF₃ | H | 493 |
| 68 | 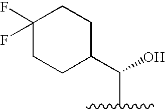 | 3-CF₃ | H | 525 |
| 69 | 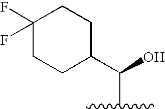 | 3-CF₃ | H | 525 |
| 70 | (CH₃)₃C—O— | 4-OCF₃ | H | 465 |
| 71 | 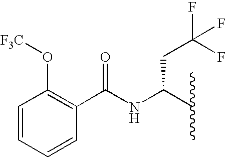 | 3-CF₃ | H | 675 |
| 72 | 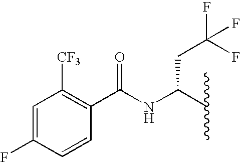 | 3-CF₃ | H | 678 |
| 73 | 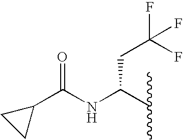 | 3-CF₃ | H | 556 |
| 74 | 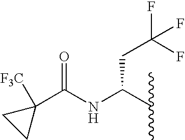 | 3-CF₃ | H | 624 |
| 75 | 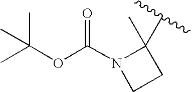 | 3-CF₃ | H | 546 |
| 76 | 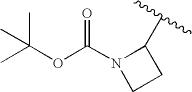 | 3-CF₃ | H | 532 |
| 77 | 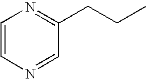 | 3-CF₃ | H | 483 |

### EXAMPLE 78

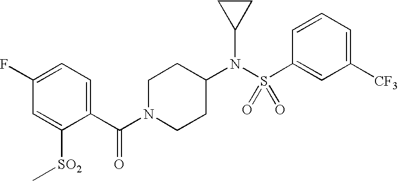

### Step 1: Preparation of 4-fluoro-2-(methylsulfonyl)benzoic acid

A 100 ml round bottom flask fitted with a stirbar and septum was charged with 4-fluoro-2-bromobenzoic acid (250 mg), sodium methylsulfonate (0.47 g), CuI (0.88 g), 5M NaOH (0.28 ml) and 15 ml methylsulfoxide [J. Org. Chem., 70: 268-274 (2005)]. After flushing with N₂, the reaction mixture was heated at 120° C. for two hours under N₂. The reaction was then cooled to room temperature and diluted with 0.5 N HCl (80 mL) and ethyl acetate (50 mL), and filtered through a plug of celite. The organic phase was separated, and the aqueous phase was extracted with ethyl acetate (30 mL). The combined organics phase was extracted with aqueous 5% KOH (25 mL) (2×). The aqueous layer was then acidified with 3N HCl and extracted with ethyl acetate (30 mL; 2×). The extracts were dried over Na₂SO₄, filtered and concentrated to give 220 mg of white solid as the desired product. ¹H NMR (CDCl₃): ¹H NMR (CDCl₃): 7.95 (m, 2H), 7.43 (m, 1H), 3.46 (s, 3H).

### Step 2: Preparation of

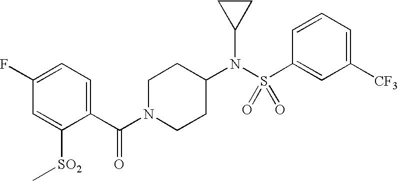

To a 15 ml vial were charged with amine (Example 1, Step 3, 49 mg), 4-fluoro-2-(methylsulfonyl)benzoic acid (30 mg), diisopropylethyl amine (72 mg), Bop reagent (benzotriazole-1-yl-oxy-tris-(dimethylamino)-phosphonium hexafluorophosphate) (74 mg) and 1 ml dimethylformamide. After reaction mixture was stirred at room temperature one hour, it was diluted with 0.5 ml methylsulfoxide, 0.5 ml H₂O and 0.1 ml trifluoroacetic acid. The resulting mixture was loaded on to reverse phase column and purified by reversed-phase HPLC with gradient solvent acetonitrile/H₂O (10%-90%) to give 60 mg desired product as fluffy white solid after lyophilizing. ¹H NMR indicating it contains a pair of rotamers in the solution at 2:1 ratio. ¹H NMR spectrum was complicated due to rotamers. ¹H NMR (CD₃OD): 8.2 (m), 8.0 (d, J=8.1 Hz, 1H), 7.8 (m), 7.5 (m), 4.7 (d, 1H major rotamer), 4.6 (d, 1H, minor rotamer), 4.2 (m, 1H), 3.2 (m, 1H) 3.31 (s, 3H, major rotamer), 3.27 (s, 3H, minor rotamer), 3.2 (d, 1H, major rotamer), 3.05 (m, 1H, minor rotamer), 2.8 (m, 1H) 1.3-2.2 (m, 5H), 0.9 (b, 2H), 0.8 (b, 2H). MS: m/e 549 (M+1)⁺.

TABLE 3

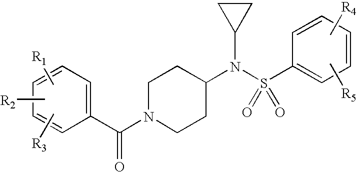

| EX- | | | | | | Mass |
|---|---|---|---|---|---|---|
| AM- | | | | | | (M + H) |
| PLES | R₁ | R₂ | R₃ | R₄ | R₅ | m/e |
| 79 | 3-CF₃ | 5-CF₃ | H | H | H | 521 |
| 80 | 2-CH₃SO₂ | H | H | 3-CF₃ | H | 531 |
| 81 | 3-CH₃SO₂ | H | H | 3-CF₃ | H | 531 |
| 82 | 4-SO₂NH₂ | H | H | 3-CF₃ | H | 532 |
| 83 | 4-CH₃SO₂ | H | H | 3-CF₃ | H | 531 |
| 84 | 3-CONH₂ | H | H | 3-CF₃ | H | 496 |
| 85 | 3-COOCH₃ | H | H | 3-CF₃ | H | 511 |
| 86 | 4-COOCH₃ | H | H | 3-CF₃ | H | 511 |
| 87 | 2-CH₃SO₂ | H | H | 4-OCF₃ | H | 547 |
| 88 | 2-CH₃SO₂ | 5-F | H | 3-CF₃ | H | 549 |
| 89 | 2-CH₃SO₂ | 4-Cl | H | 3-CF₃ | H | 566 |
| 90 | 2-CH₃SO₂ | 4-Cl | H | 3-OCF₃ | H | 582 |
| 91 | 2-CH₃SO₂ | 5-F | H | 3-OCF₃ | H | 565 |
| 92 | 2-CH₃SO₂ | 6-F | H | 3-CF₃ | H | 549 |
| 93 | 2-CH₃SO₂ | 4-F | H | 3-CF₃ | H | 549 |
| 94 | 2-CH₃SO₂ | 4-F | 5-F | 3-CF₃ | H | 567 |
| 95 | 2-CH₃SO₂ | 6-OCF₃ | H | 3-CF₃ | H | 615 |
| 96 | 2-CH₃SO₂ | H | H | 2-CF₃ | H | 531 |
| 97 | 2-CH₃SO₂ | 5-F | H | 2-CF₃ | H | 549 |
| 98 | 2-CH₃SO₂ | H | H | 2-OCF₃ | H | 547 |
| 99 | 2-CH₃SO₂ | 5-F | H | 2-OCF₃ | H | 565 |
| 100 | 2-CH₃CO | H | H | 3-CF₃ | H | 495 |
| 101 | 2-CH₃SO₂ | 5-F | H | 2-Br | 3-CF₃ | 627 |
| 102 | 2-CH₃SO₂ | H | H | 2-Cl | 3-CF₃ | 566 |
| 103 | 2-CH₃SO₂ | 5-F | H | 2-Cl | 3-CF₃ | 583 |
| 104 | 2-CONH₂ | H | H | 3-CF₃ | H | 496 |
| 105 | 2-COOCH₃ | H | H | 3-CF₃ | H | 511 |
| 106 | 2-CONHCH₃ | H | H | 3-CF₃ | H | 510 |
| 107 | 2-CH₃SO₂ | H | H | 3-CH₃SO₂ | H | 541 |
| 108 | 2-CH₃SO₂ | 5-F | H | 3-CH₃SO₂ | H | 559 |
| 109 | 2-CH₃SO₂ | 3-F | H | 3-CF₃ | H | 549 |
| 110 | 2-CH₃SO₂ | 5-CN | H | 3-CF₃ | H | 556 |
| 111 | 2-CH₃SO₂ | 5-F | H | 3-CF₃ | 5-CF₃ | 618 |
| 112 | 2-CH₃SO₂ | 5-F | H | 3-CF₃ | 5-F | 567 |

### EXAMPLE 113

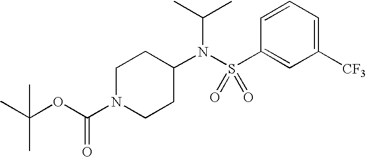

### Step 1: Preparation of

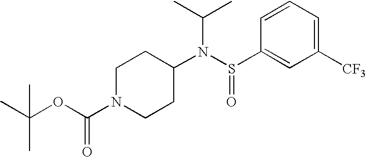

To a solution of tert-butyl 4-(isopropylamino)piperidine-carboxylate (0.25 g) (prepared using similar chemistry described in Step 1, Example 1) in CH₂Cl₂ (10 ml) were added 3-trifluoromethylsulfinylchloride (1.4 mL; 0.8 M in THF solution) [J. Org. Chem. USSR (Engl. Transl.) 13: 2086-2087 (1977)], N,N-dimethylaminopyridine (2 grains), diisopropylethylamine (0.71 mL). The resulting reaction mixture was stirred at room temperature two hours, and then diluted with ether (50 mL), washed with 1N HCl (50 mL) and then 50 ml 5% KOH. The organic phase was washed with water, dried over Na₂SO₄, filtered and concentrated. The residue obtained was purified by silica gel chromatography using ethyl acetate/hexane (1:3) to afford the titled compound as a white solid (110 mg). ¹H NMR (CDCl₃): 7.94 (s, 1H), 7.87 (d, J=7.8 Hz, 1H), 7.76 (d, J=8.0 Hz, 1H), 7.66 (t, J=7.8 Hz, 1H), 4.2 (b, 2H), 3.57 (qint, J=4.6 Hz, 1H), 3.23 (m, 1H), 2.74 (b, 1H), 2.60 (b, 1H), 2.15 (m, 1H), 1.95 (m, 1H), 1.75 (m, 1H), 1.48 (s, 9H), 1.43 (d, J=5.6 Hz, 3H), 1.15 (b, 3H). MS: m/e 435 (M+1)⁺.

### Step 2: Preparation of

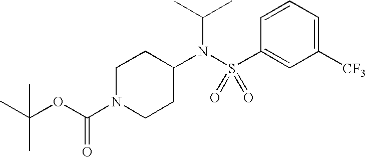

A 25 ml round bottom flask was charged with sulfinamide (110 mg, Step 1), metachloroperbenzoic acid (156 mg, 60%) and CH₂Cl₂ (5 ml), and the resulting reaction mixture was stirred at room temperature overnight. The reaction was diluted with ether, washed with 5% KOH, then brine. The organic layer was dried over Na₂SO₄, filtered and concentrated in vacuo. The crude product was purified by reversed-phase HPLC (10-90% acetonitrile/H₂O containing 0.1% trifluoroacetic acid) to give 110 mg of the titled compound as a white solid. ¹H NMR (CDCl₃): 8.15 (s, 1H), 8.08 (d, J=7.3 Hz, 1H), 7.83 (d, J=7.3 Hz, 1H), 7.67 (t, J=7.7 Hz, 1H), 4.2 (b, 2H), 3.8 (qint, J=6.8 Hz, 1H), 3.4 (m, 1H), 2.75 (b, 1H), 2.05 (m, 2H), 1.95 (b, 1H), 1.75 (b, 1H), 1.75 (m, 1H), 1.48 (s, 9H), 1.3 (b, 3H). MS: m/e 451 (M+1)⁺.

TABLE 4

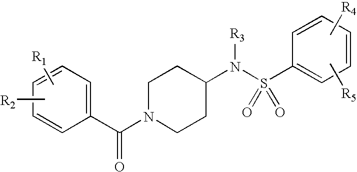

| | | | | | | Mass |
|---|---|---|---|---|---|---|
| | | | | | | (M + H) |
| EXAMPLES | R₁ | R₂ | R₃ | R₄ | R₅ | m/e |
| 114 | 3-CF₃ | 4-F | H | 3-CF₃ | H | 499 |
| 115 | 2-CH₃SO₂ | H | CH₃ | 3-CF₃ | H | 505 |
| 116 | 2-CH₃SO₂ | H | Ethyl | 3-CF₃ | H | 519 |
| 117 | 2-OCF₃ | H | 4- | 3-CF₃ | H | 602 |
| | | | (OCH₃)Ph | | | |
| 118 | 2-OCF₃ | H | H | 3-CF₃ | H | 497 |
| 119 | 2-CH₃SO₂ | H | H | 3-CF₃ | H | 491 |
| 120 | 2-CH₃SO₂ | 5-F | H | 3-CF₃ | H | 509 |
| 121 | 2-CH₃SO₂ | H | Isopropyl | 3-CF₃ | H | 533 |
| 122 | 2-CH₃SO₂ | 5-F | Isopropyl | 3-CF₃ | H | 551 |
| 123 | 2-CH₃SO₂ | 4-F | Isopropyl | 3-CF₃ | H | 551 |

### EXAMPLE 124

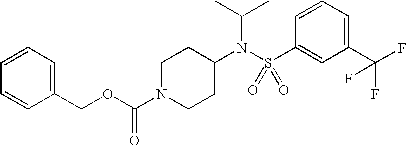

To a mixture of the amine (from Example 1, Step 3) (62 mg, 0.175 mmol) and diisopropylethyl amine (110 mg) in tetrahydrofuran (1 ml) was added benzyl chloridocarbonate (20 mg, 0.12 mmol). The reaction mixture was stirred at ambient temperature for 18 h. The crude mixture (without any work-up) was loaded on to the reverse phase column (C18, YMC) and purified by HPLC using CH₃CN/H₂O (containing 0.1% trifluoroacetic acid) gradient (10%-90%) to give the titled compound as a white solid (50 mg).

¹H NMR (CDCl₃): 8.15 (s, 1H), 8.08 (d, J=8 Hz, 1H), 7.8 (d, J=8 Hz, 1H), 7.7 (t, J=16 Hz, 1H), 7.3-7.4 (m, 4H), 7.2 (m, 1H), 4.2-4.4 (b, 4H), 4.05 (m, 1H), 2.65-2.85 (b, 4H), 1.9 (m, 1H), 1.6 (s, 2H), 0.82-1.0 (B, 2H), 0.8 (d, J=4, 2H) MS (ESI): m/e 483 (M+1)⁺

### EXAMPLE 125

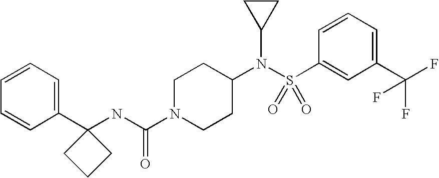

To a mixture of the amine (from Step 3, Example 1) (62 mg, 0.175 mmol) and diisopropylethyl amine (74 mg, 0.57 mmol) in tetrahydrofuran (2 mL) was added 1-phenyl cyclobutyl isocyanate (20 mg, 0.12 mmol). The resulting reaction mixture was stirred at room temperature for 1 h and then quenched with 1N HCl (5 mL), extracted with ethyl acetate (5 mL). The organic layer was concentrated, and the crude product obtained was purified by reverse phase column (C₁₋₈, YMC) using CH₄CN/H₂O (containing 0.1% trifluoroacetic acid) as the gradient solvents (10%-90%) to give the titled compound as a white solid (55 mg). ¹H NMR (CDCl₃): 8.15 (s, 1H), 8.08 (d, J=7 Hz, 1H), 7.88 (d, J=8 Hz, 1H), 7.7 (t, J=16 Hz, 1H), 7.45 (d, J=8 Hz, 2H), 7.3 (t, J=9 Hz, 2H), 7.2 (t, J=8 Hz, 1H), 5.19b, 1H), 4.0 (m, 3H), 2.78 (t, J=12 Hz, 4H), 2.63 (m, 2H), 2.53 (m, 2H), 2.1 (m, 1H), 1.96 (m, 1H), 1.85 (m, 4H), 0.94 (s, 2H), 0.78 (d, J=7 Hz, 2H). MS (ESI): m/e 522 (M+1)⁺

### EXAMPLE 126

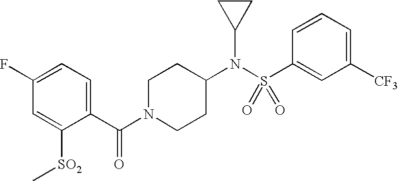

### Step 1: Preparation of

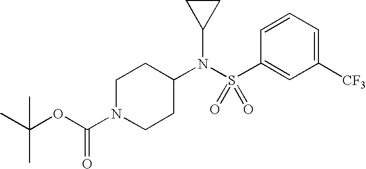

A 250 ml round bottom flask was charged with the piperidine sulfonamide (from Step 2, Example 1) (1.1 g), NaIO₄ (1.07 g), EtOAc (20 ml) and H₂O (20 ml). The flask was then flushed with N₂ and RuCl₃.H₂O was added, and the mixture was stirred at room temperature overnight. The reaction mixture was diluted with ethyl acetate (50 mL), filtered through a pad of celite. The filtrate was washed with H₂O (50 mL), dried over Na₂SO₄ and concentrated. The crude product obtained was purified by silica gel chromatography using ethyl acetate/hexane (1:4 to 1:3) to give desired protected piperidone. The piperiridone thus obtained was treated with trifluoroacetic acid (2 mL) at room temperature for 2 h. The reaction was carefully treated with 5% KOH and extracted with ethyl acetate (30 mL×2). The organic phase was washed with water, dried over Na₂SO₄ and concentrated to give the titled piperidone compound as a white solid. ¹H NMR (CD₃OD): 8.19 (d, J=8.0 Hz, 1H), 8.16 (s, 1H), 8.01 (d, J=7.8 Hz, 1H), 7.85 (t, J=7.8 Hz, 1H), 4.36 (m, 1H), 3.25 (m, 2H), 2.7 (dd, J=17 Hz, J=7.6 Hz, 1H), 2.35 (m, 1H), 2.1 (m, 2H), 1.80 (d, J=12.8 HZ, 1H), 0.94 (m, 2H), 0.80 (m, 2H). MS: m/e 363 (M+1)⁺.

### Step 2: Preparation of

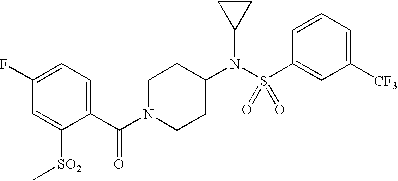

To a solution of the piperidone (from Step 1, above) (60 mg) in dry dimethylformamide (2 mL) was added NaH (13.2 mg, 60% with oil) followed by trifluoromethylbenzylbromide (60 mg). The reaction was stirred at room temperature for 5 minutes, and then quenched by water (0.5 mL), trifluoroacetic acid (0.1 mL) and methyl sulfoxide (1 mL). The mixture was loaded on to reverse phase column (C₁₈, YMC) and purified by HPLC using CH₃CN/H₂O (containing 0.1% trifluoroacetic acid) as the gradient solvents (10%-90%) to give the title compound as a white solid (50 mg). ¹H NMR (CD₃OD): 8.21 (d, J=8.0 Hz, 1H), 8.18 (s, 1H), 8.02 (d, J=8 Hz, 1H), 7.86 (t, J=7.9 Hz, 1H), 7.72 (d, J=8 Hz, 1H), 7.63 (t, J=7.8 Hz, 1H), 7.46 (t, J=7.7 Hz, 1H), 7.35 (t, J=7.8 Hz, 1H), 5.0 (d, J=16.4, 1H), 4.6 (d, J=16.4, 1H), 4.45 (m, 1H), 3.35 (m, 1H), 3.25 (m, 1H), 2.90 (dd, J=16.9 Hz, J=1.9 Hz, 1H), 2.45 (m, 1H), 2.25 (m, 1H), 2.12 (m, 1H), 1.90 (m, 1H), 0.96 (m, 2H), 0.85 (m, 2H). MS: m/e 321 (M+1)⁺.

Further examples of this invention are summarized in the following table:

TABLE 5

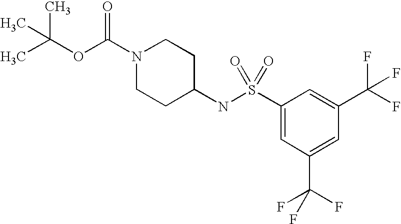

EXAMPLE 127
Mass (M + H) m/e: 477

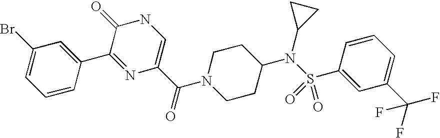

EXAMPLE 128
Mass (M + H) m/e: 626

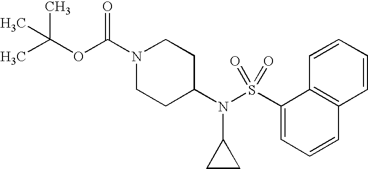

EXAMPLE 129
Mass (M + H) m/e: 431

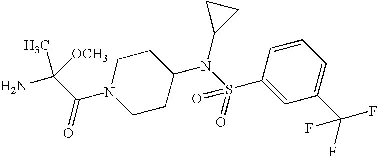

EXAMPLE 130
Mass (M + H) m/e: 434

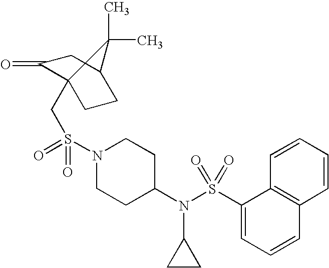

EXAMPLE 131
Mass (M + H) m/e: 545

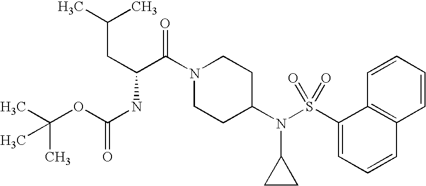

EXAMPLE 132
Mass (M + H) m/e: 544

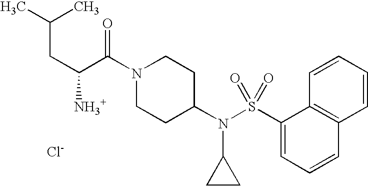

EXAMPLE 133
Mass (M + H) m/e: 444

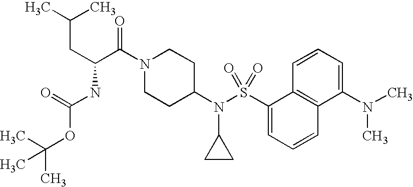

EXAMPLE 134
Mass (M + H) m/e: 587

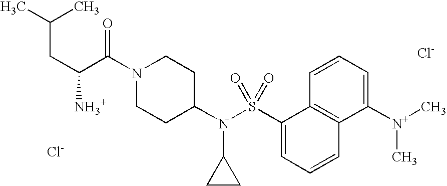

EXAMPLE 135
Mass (M + H) m/e: 487

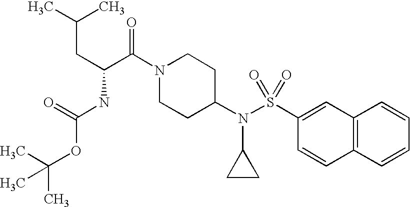

EXAMPLE 136
Mass (M + H) m/e: 544

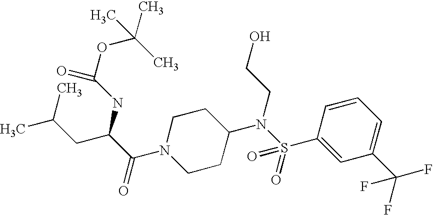

EXAMPLE 137
Mass (M + H) m/e: 566

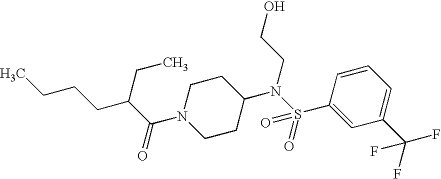

EXAMPLE 138
Mass (M + H) m/e: 479

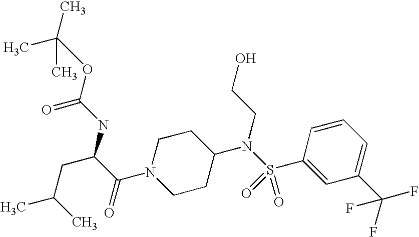

EXAMPLE 139
Mass (M + H) m/e: 522

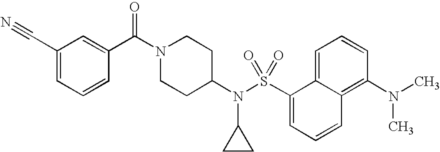

EXAMPLE 140
Mass (M + H) m/e: 503

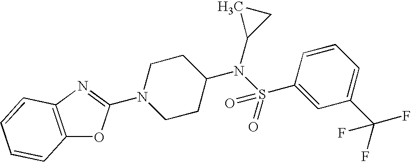

EXAMPLE 141
Mass (M + H) m/e: 482

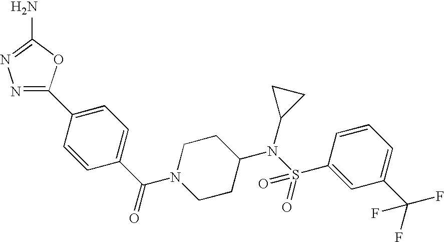

EXAMPLE 142
Mass (M + H) m/e: 536

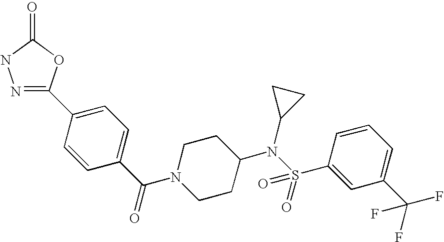

EXAMPLE 143
Mass (M + H) m/e: 537

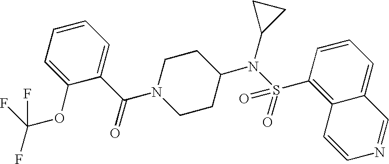

EXAMPLE 144
Mass (M + H) m/e: 520

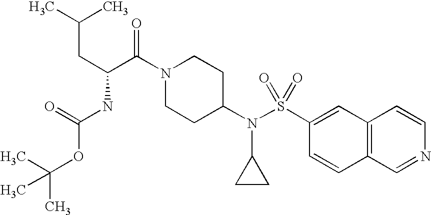

EXAMPLE 145
Mass (M + H) m/e: 545

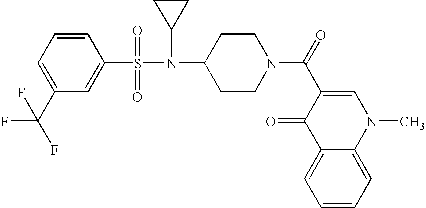

EXAMPLE 146
Mass (M + H) m/e: 534

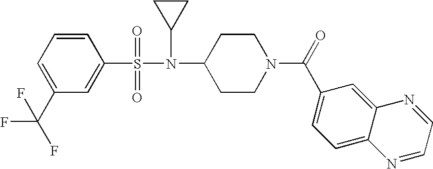

EXAMPLE 147
Mass (M + H) m/e: 504

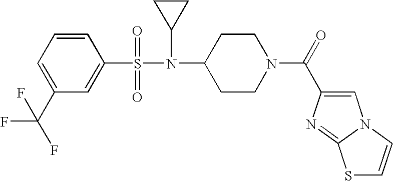

EXAMPLE 148
Mass (M + H) m/e: 499

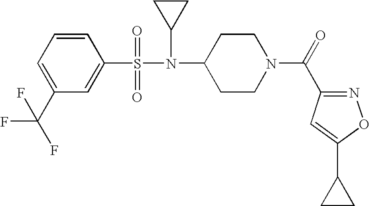

EXAMPLE 149
Mass (M + H) m/e: 484

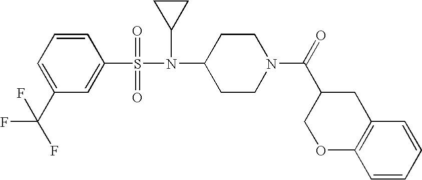

EXAMPLE 150
Mass (M + H) m/e: 509

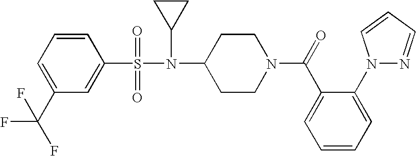

EXAMPLE 151
Mass (M + H) m/e: 519

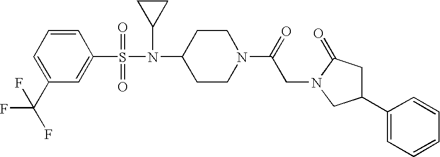

EXAMPLE 152
Mass (M + H) m/e: 550

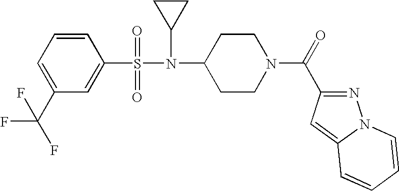

EXAMPLE 153
Mass (M + H) m/e: 493

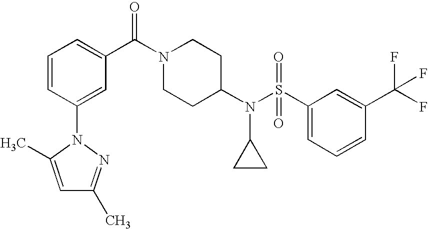

EXAMPLE 154
Mass (M + H) m/e: 547

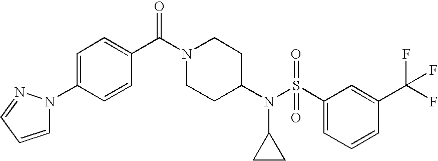

EXAMPLE 155
Mass (M + H) m/e: 518

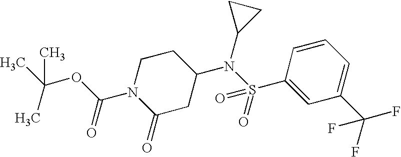

EXAMPLE 156
Mass (M + H) m/e: 463

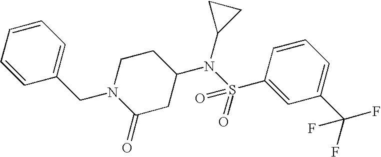

EXAMPLE 157
Mass (M + H) m/e: 453

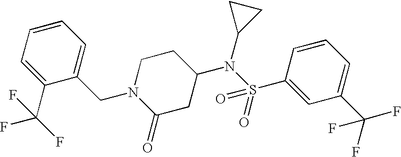

EXAMPLE 158
Mass (M + H) m/e: 521

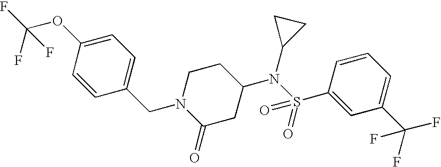

EXAMPLE 159
Mass (M + H) m/e: 537

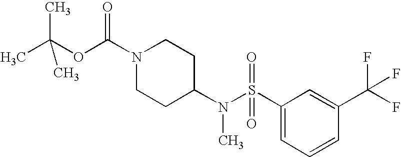

EXAMPLE 160
Mass (M + H) m/e: 423

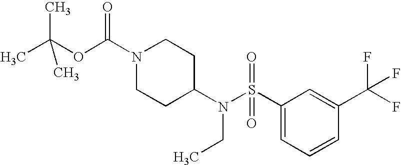

EXAMPLE 161
Mass (M + H) m/e: 437

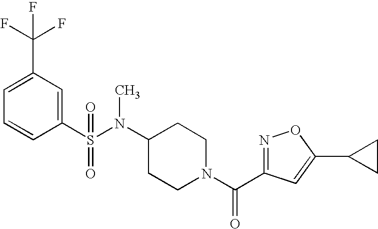

EXAMPLE 162
Mass (M + H) m/e: 458

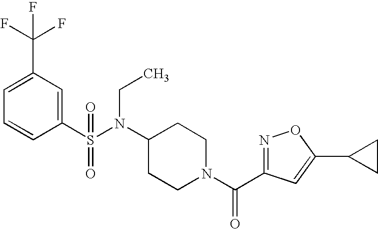

EXAMPLE 163
Mass (M + H) m/e: 472

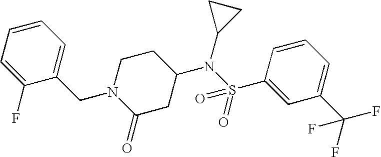

EXAMPLE 164
Mass (M + H) m/e: 471

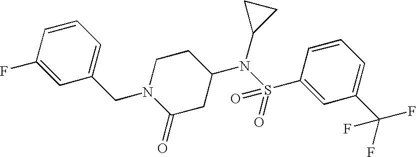

EXAMPLE 165
Mass (M + H) m/e: 471

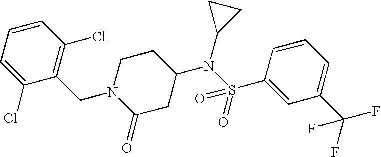

EXAMPLE 166
Mass (M + H) m/e: 522

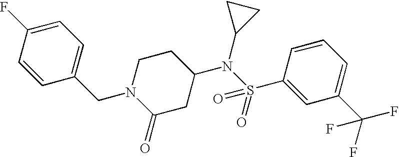

EXAMPLE 167
Mass (M + H) m/e: 471

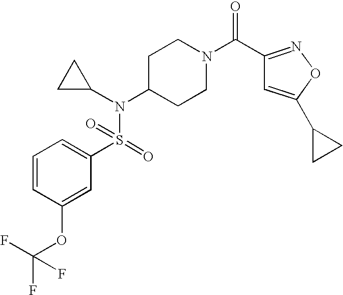

EXAMPLE 168
Mass (M + H) m/e: 500

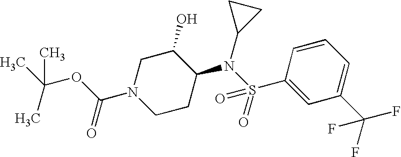

EXAMPLE 169
Mass (M + H) m/e: 465

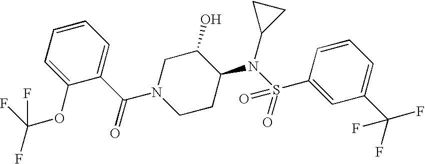

EXAMPLE 170
Mass (M + H) m/e: 553

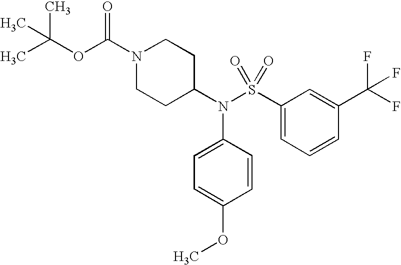

EXAMPLE 171
Mass (M + H) m/e: 515

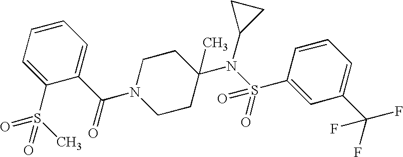

EXAMPLE 172
Mass (M + H) m/e: 545

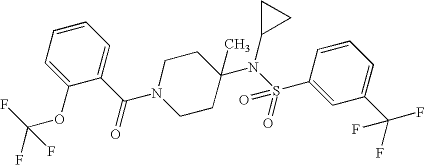

EXAMPLE 173
Mass (M + H) m/e: 551

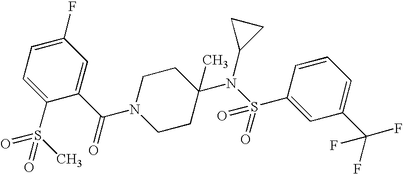

EXAMPLE 174
Mass (M + H) m/e: 563

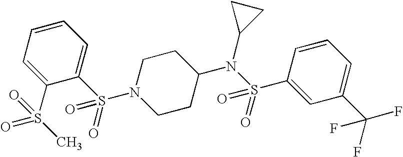

EXAMPLE 175
Mass (M + H) m/e: 567

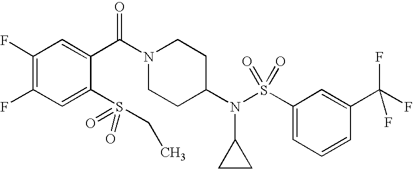

EXAMPLE 176
Mass (M + H) m/e: 581

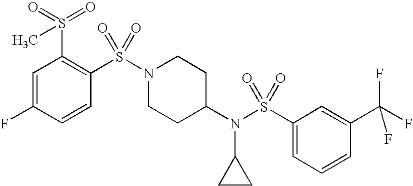

EXAMPLE 177
Mass (M + H) m/e: 585

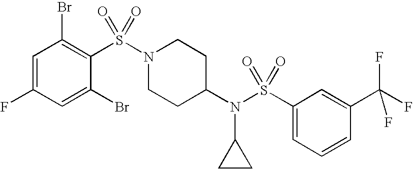

EXAMPLE 178
Mass (M + H) m/e: 604

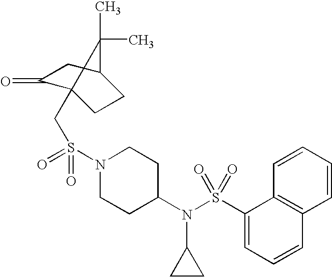

EXAMPLE 179
Mass (M + H) m/e: 545

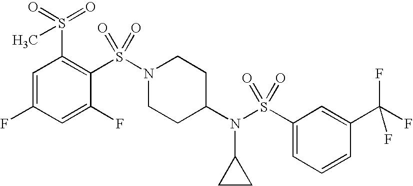

EXAMPLE 180
Mass (M + H) m/e: 603

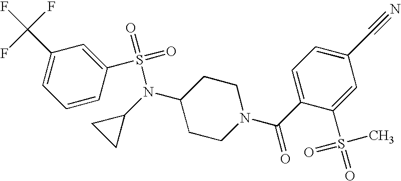

EXAMPLE 181
Mass (M + H) m/e: 556

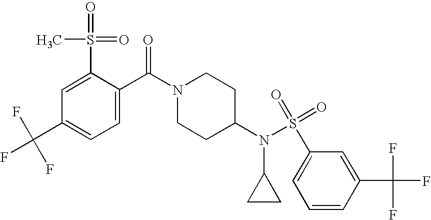

EXAMPLE 182
Mass (M + H) m/e: 599

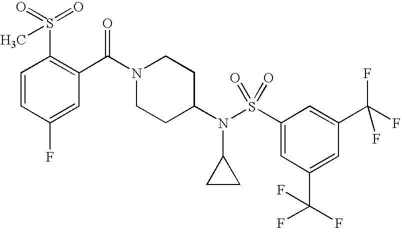

EXAMPLE 183
Mass (M + H) m/e: 617

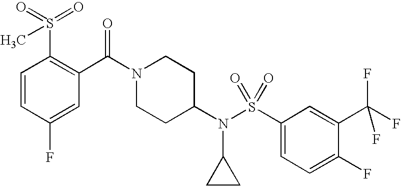

EXAMPLE 184
Mass (M + H) m/e: 567

1. A compound represented by Formula I: 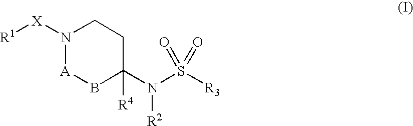
or pharmaceutically acceptable salts thereof, wherein:
X is —CH(R^{a})—, C═O, (C═O)O, NH(C═O), SO₂ or —NHSO₂;
A and B each independently is CH₂, CH—C₁-C₄alkyl, CH—OH or CO;
R¹ is:
(a) C₁-C₈ alkyl,
(b) C₃-C₆ cycloalkyl,
(c) C₃-C₆ cycloalkyl-C(O)O—C₀-C₆alkyl,
(d) aryl-heteroaryl,
(e) N-heterocycle,
(f) N-aryl,
(g) C₁-C₄ alkyl-COOH,
(h) C₁-C₄alkl-C(O)—N—C₁-C₄alkyl-R^{a},
(i) N—C₁-C₄ alkyl(aryl)(COOH),
(j) C₁-C₄alkyl(N—C(O)-heterocycle)(C₀-C₄alkyl-aryl),
(k) C₁-C₄alkyl(N—C(O)O—C₁-C₄alkyl)(C₀-C₄-alkyl-C₀-C₄ perfluoroalkyl),
(l) C₁-C₄alkyl-N—C(O)-aryl,
(m) C₁-C₄alkyl-N—C(O)—C₃-C₆ cycloakyl, or
(n) O—R^{a}
(o) C₀-C₄ alkyl-aryl, where said aryl is substituted with one or more substituents selected from heteroaryl, N—C₁-C₄alkyl-C(O)R^{a}, C(O)—N—C₁-C₄alkyl-R^{a}, SO₂R^{a}, C(O)R^{a}, C(O)O—R^{a}, and optionally substituted with one or more substituents selected from halogen, aryl, C₁-C₆ alkyl, C₁-C₆ haloalkyl, alkoxy, NR^{a}, O—CF₃, CN, C₃-C₆ cycloalkyl, or OH;
(p) C₀-C₄ alkyl-heteroaryl, where said heteroaryl is substituted with one or more substituents selected from heteroaryl, N—C₁-C₄alkyl-C(O)R^{a}, C(O)—N—C₁-C₄alkyl-R^{a}, SO₂R^{a}, C(O)R^{a}, C(O)O—R^{a}, and optionally substituted with one or more substituents selected from halogen, aryl, C₁-C₆ alkyl, C₁-C₆ haloalkyl, alkoxy, NR^{a}, O—CF₃, CN, C₃-C₆ cycloalkyl, or OH;
R² is:
(a) H,
(b) C₁-C₆-alkyl,
(c) C₁-C₆—OH,
(d) C₃-C₆ cycloalkyl, or
(e) C₀-C₆ alkyl-aryl, wherein said aryl is optionally substituted with one or more substituents selected from alkyl, O—C₁-C₄ alkyl, and halogen,
or the N to which R² is attached joins with two adjacent atoms to form a heterocycle, of N and the SO₂ to which it is attached joins to form a heterocycle, wherein said heterocycle is optionally substituted with one or more substituents selected from O and CF₃,
R³ is:
(d) aryl, or
(e) heteroaryl,
said aryl is optionally substituted with one or more substituents selected from CF₃, CN, halogen, C₁-C₄—OH, N—R^{a}, O—R^{a}, and N—C(O)—R^{a}, and said heteroaryl is optionally substituted with one or more substituents selected from S—R^{a}, heterocycle, and C(O)—N—R^{a};
R⁴ is:
(a) H,
(b) —C₁-C₄-alkyl, or
(f) OH;
R^{a} is:
(a) H,
(b) C₁-C₆ alkyl,
(c) —C₀-C₆-alkyl-heterocycloalkyl,
(d) —C₁-C₆-alkoxy,
(e) NH2, or
(f) —C₀-C₆-aryl;
R^{b} is:
(a) H,
(b) C₁-C₆ alkyl,
(c) OH,
(d) —C₁-C₆-alkoxy,
(e) NH₂, or
(f) NH—C₁-C₄-alkyl or N(C₁-C₄-alkyl)₂.

2. The compound of claim 1, or a pharmaceutically acceptable salt thereof, wherein X is selected from C═O, —CO—NH—, SO₂ and CH₂.

3. The compound of claim 1, or a pharmaceutically acceptable salt thereof, wherein A and B each independently is CH₂.

4. The compound of claim 1, or a pharmaceutically acceptable salt thereof, wherein
R¹ is phenyl substituted with one or more substituents selected from halogen, CF₃, O—R^{a}, and SO₂—R^{a}.

5. The compound of claim 1, or a pharmaceutically acceptable salt thereof, wherein R² is cyclopropyl.

6. The compound of claim 1, or a pharmaceutically acceptable salt thereof, wherein R³ is phenyl substituted with one or more CF₃.

7. The compound of claim 1, or a pharmaceutically acceptable salt thereof, wherein R⁴ is hydrogen.

8. The compound of claim 1 of the Formula Ia: 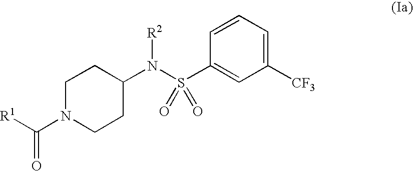
or a pharmaceutically acceptable salt thereof.

9. The compound of claim 1 of the Formula Ib: 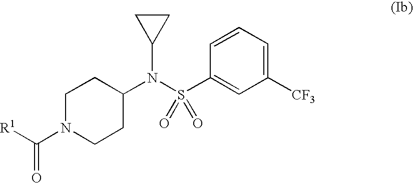
or a pharmaceutically acceptable salt thereof.

10. The compound of claim 1 of the Formula Ic: 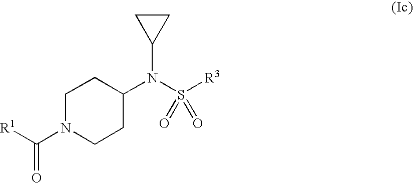
or a pharmaceutically acceptable salt thereof, wherein R³ is phenyl optionally substituted with one or more substituents selected from halogen, CF₃ and O—R^{a}.

11. The compound of claim 1 of the Formula Id: 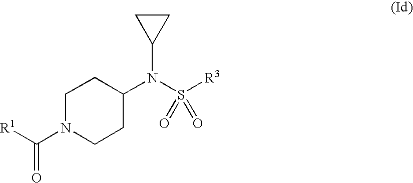
or a pharmaceutically acceptable salt thereof, wherein R¹ is C₁-C₆ alkyl, C₀-C₄ alkyl-aryl, or heteroaryl, wherein said aryl and heteroaryl each is independently optionally substituted with one or more substituents selected from SO₂—R^{a}, SO₂NH₂, CONH₂, CONHCH₃, and COOCH₃.

12. The compound of claim 1 of the Formula Ie: 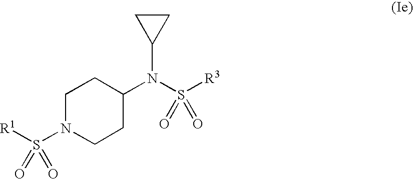
or a pharmaceutically acceptable salt thereof, wherein R¹ is C₀-C₄ alkyl-aryl, wherein said aryl is optionally substituted with one or more substituents selected from SO₂—R^{a}, SO₂NH₂, CONH₂, CONHCH₃, and COOCH₃.

13. A compound selected from: 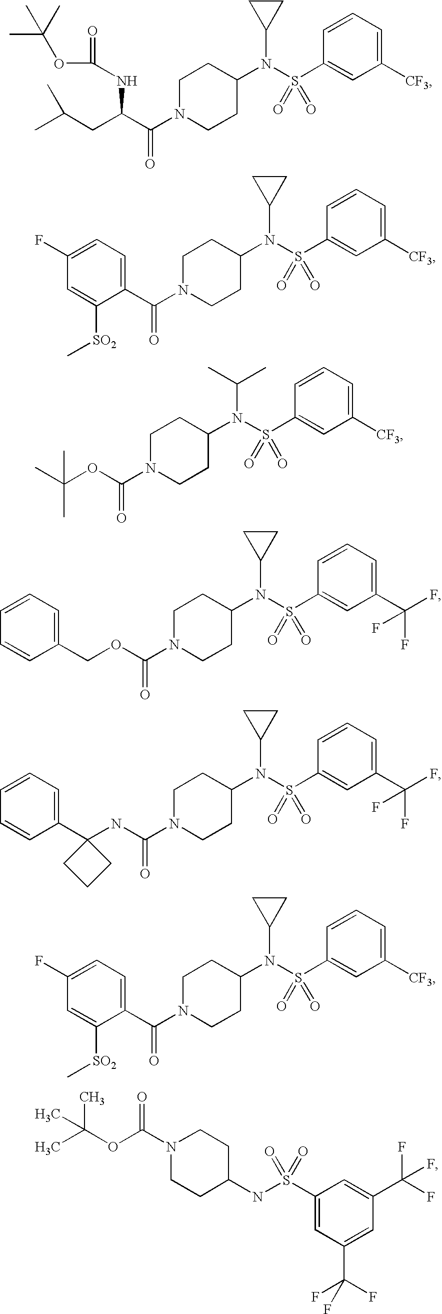 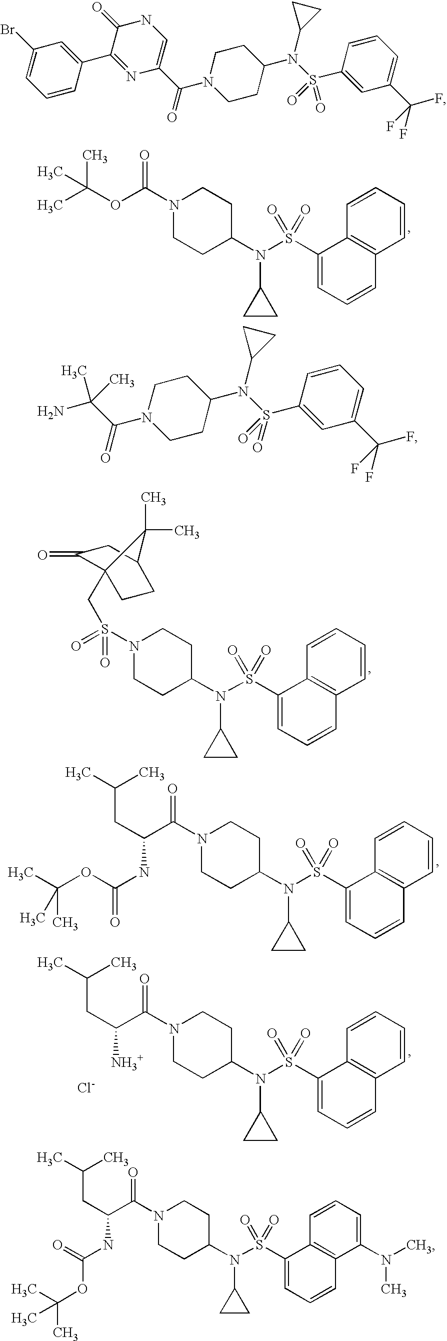 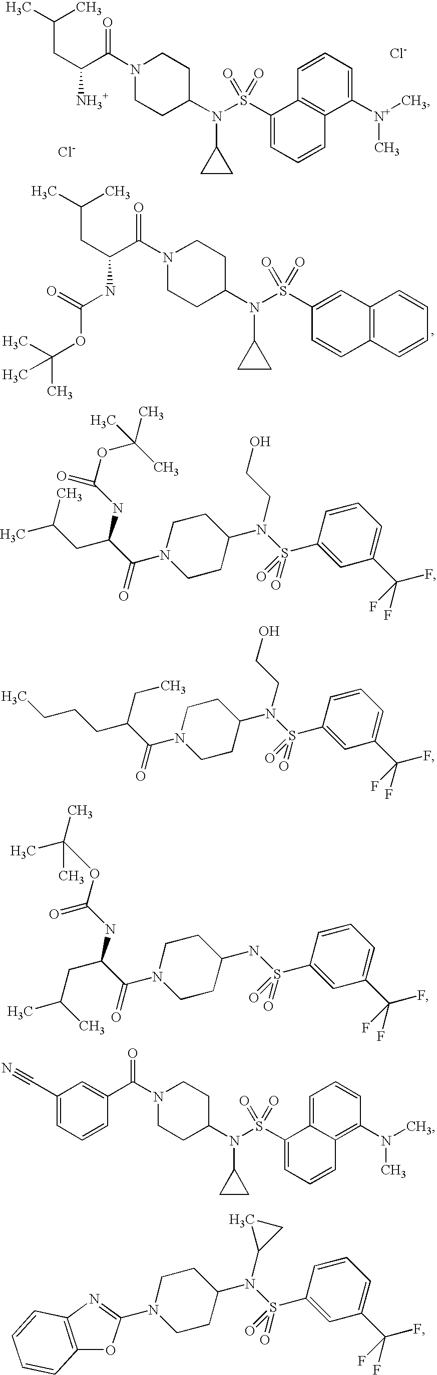 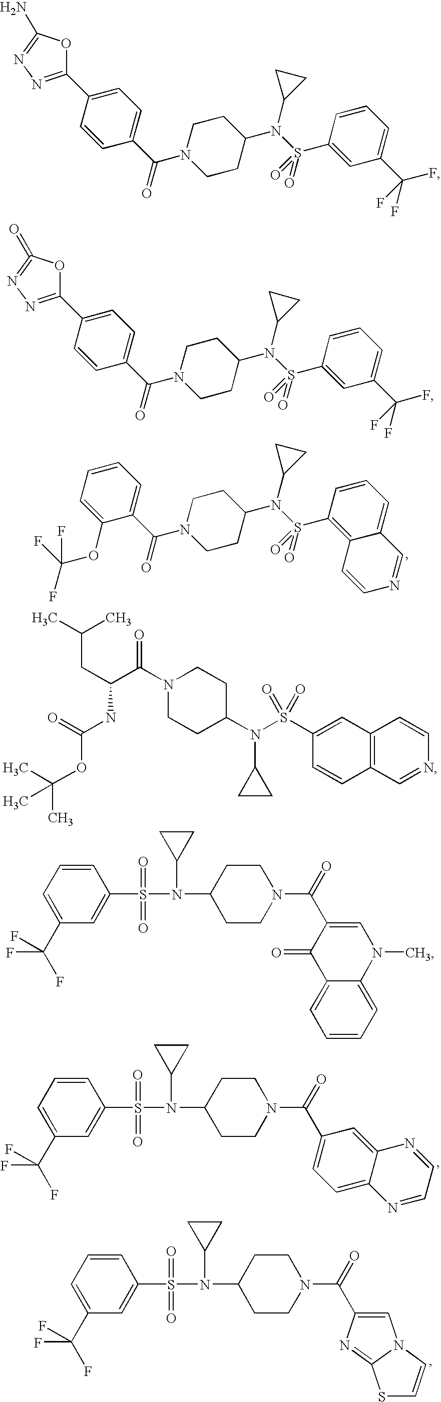 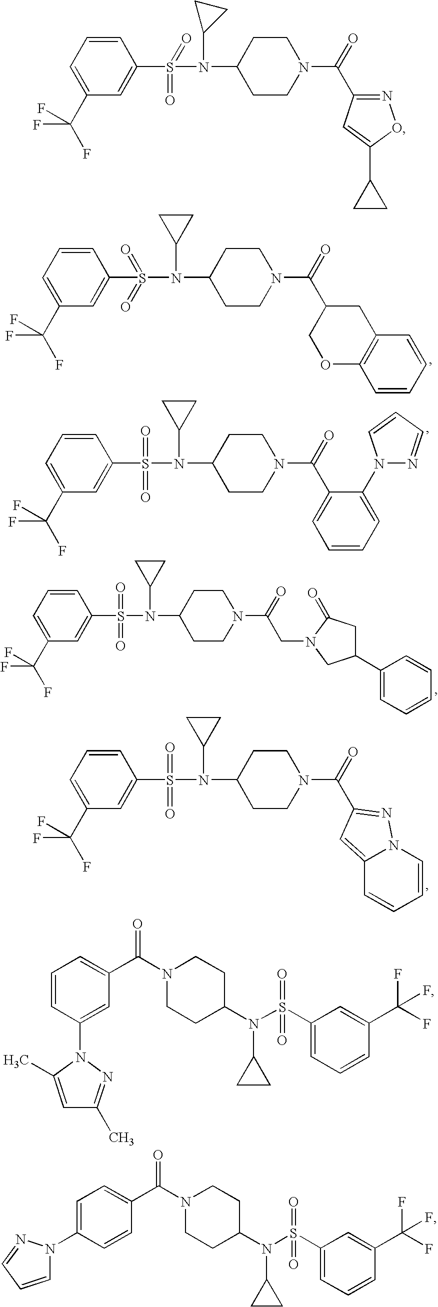 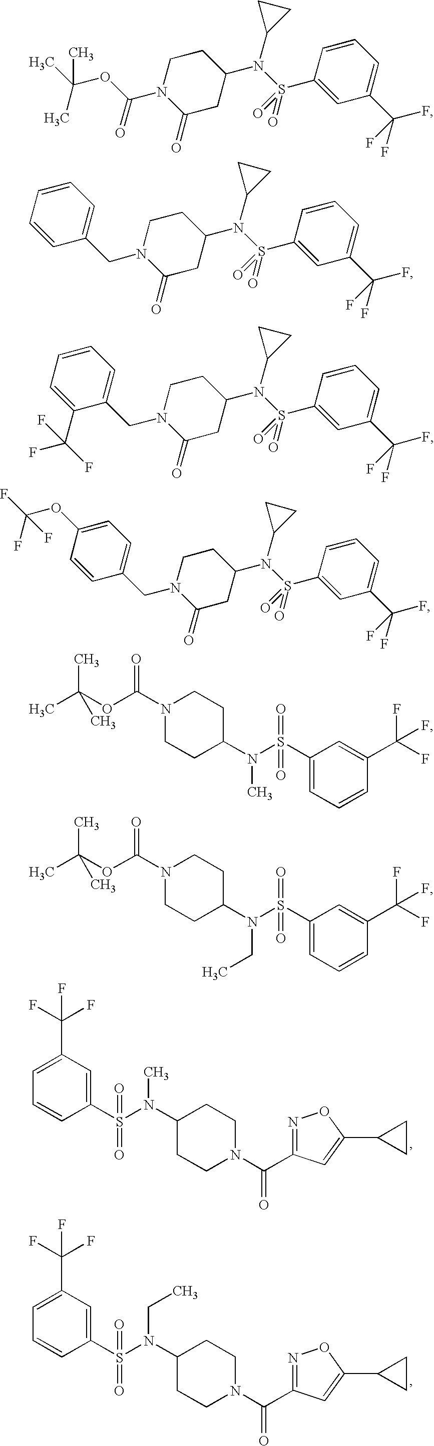 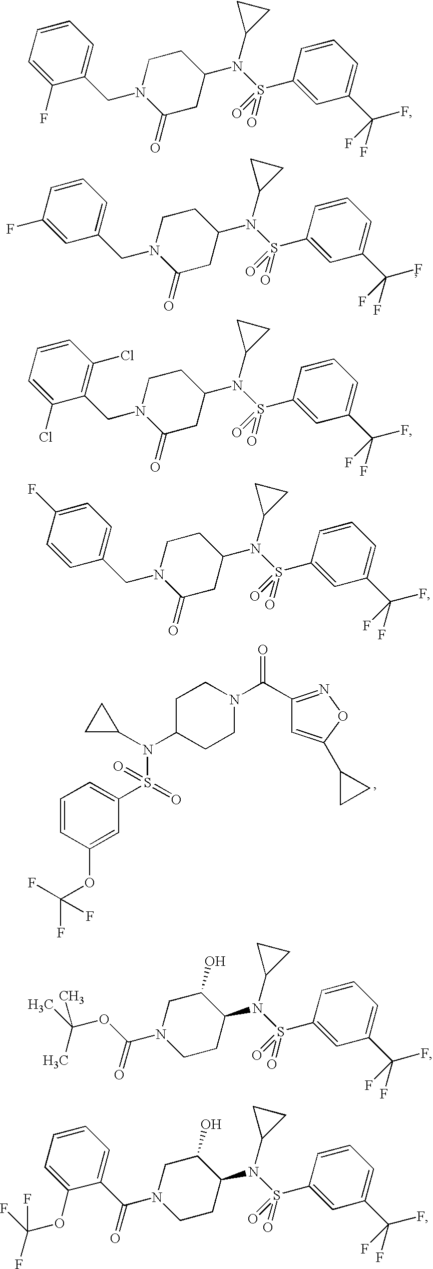 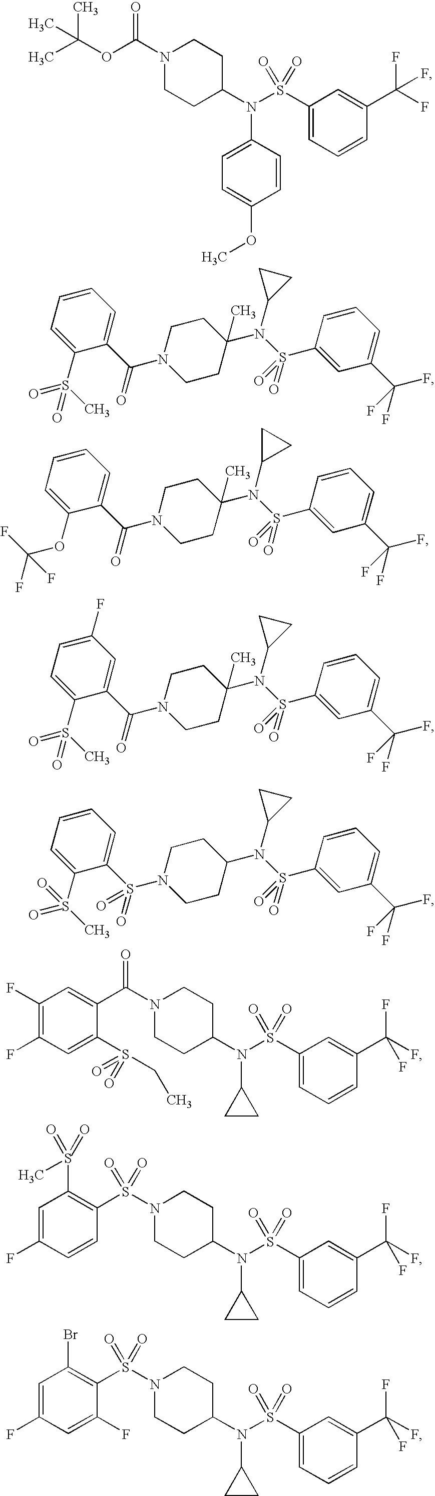 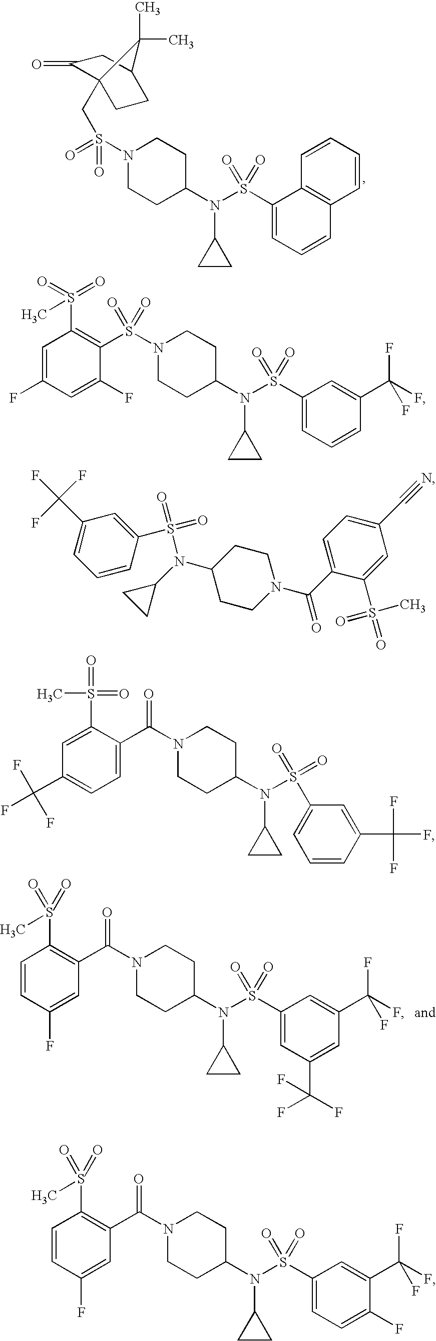
or a pharmaceutically acceptable salt or an individual diastereomer thereof.

14. The compound of claim 1 of the formula: 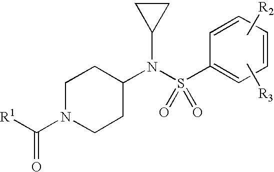
wherein R₁, R₂ and R₃ are selected from a single row in the table:

| R₁ | R₂ | R₃ |
|---|---|---|
| HOOCCH₂CH₂— | 4-Cl | H |
| Ph-NH— | 3-CF₃ | H |
| 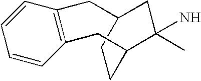 | 3-CF₃ | H |
| 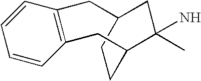 | H | H |
| 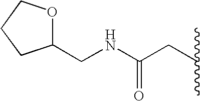 | 3-CF₃ | H |
| 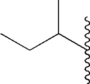 | 3-CF₃ | H |
| 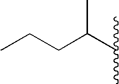 | 3-CF₃ | H |
| 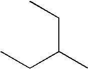 | 3-CF₃ | H |
| 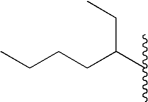 | 3-CF₃ | H |
| (CH₃)₃C— | 3-CF₃ | H |
| 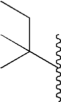 | 3-CF₃ | H |
| 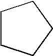 | 3-CF₃ | H |
| 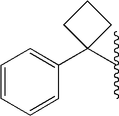 | 3-CF₃ | H |
| 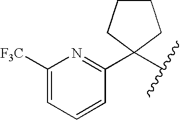 | 3-CF₃ | H |
| (CH₃)₃C—O— | 3-CF₃ | 5-CF₃ |
| 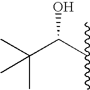 | 3-CF₃ | H |
| 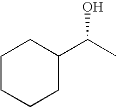 | 3-CF₃ | H |
| 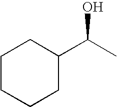 | 3-CF₃ | H |
| 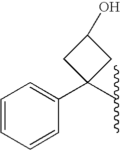 | 3-CF₃ | H |
| 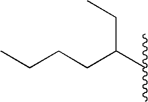 | 3-CF₃ | 5-CF₃ |
| 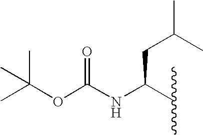 | 3-CF₃ | H |
| 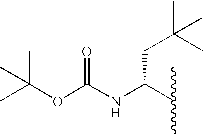 | 3-CF₃ | H |
| 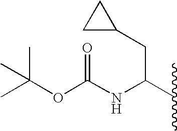 | 3-CF₃ | H |
| 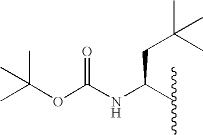 | 3-CF₃ | H |
| (CH₃)₃C—O— | 2-Br | H |
| (CH₃)₃C—O— | 2-OCH₃ | 3-Br |
| CH₃O—CH₂CH₂—O— | 3-CF₃ | H |
| CH₃CH₂CH₂CH₂CH₂—O— | 3-CF₃ | H |
| 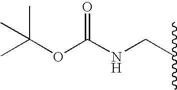 | 3-CF₃ | H |
| 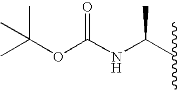 | 3-CF₃ | H |
| 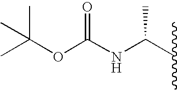 | 3-CF₃ | H |
| 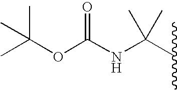 | 3-CF₃ | H |
| 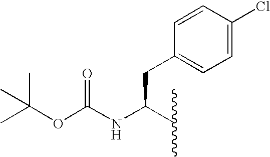 | 3-CF₃ | H |
| 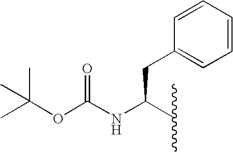 | 3-CF₃ | H |
| 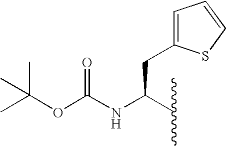 | 3-CF₃ | H |
| 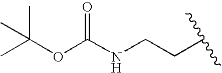 | 3-CF₃ | H |
| 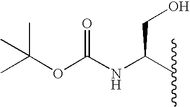 | 3-CF₃ | H |
| 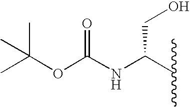 | 3-CF₃ | H |
| 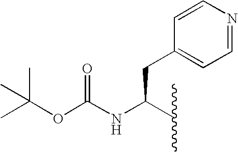 | 3-CF₃ | H |
| (CH₃)₃CH₂—O— | 3-CF₃ | H |
| (CH₃)₃—O— | 3-Cl | 5-Cl |
| H | 3-Cl | 5-Cl |
| H | 3-CF₃ | 5-CF₃ |
| 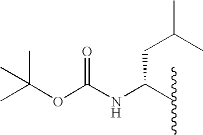 | 3-Br | H |
| 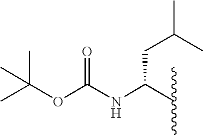 | 3-Cl | 5-Cl |
| 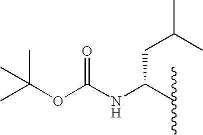 | 2-OCH₃ | 5-Br |
| 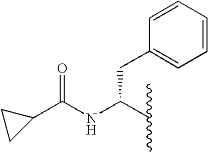 | 3-CF₃ | H |
| 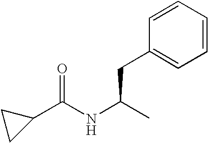 | 3-CF₃ | H |
| 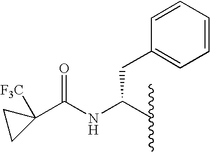 | 3-CF₃ | H |
| 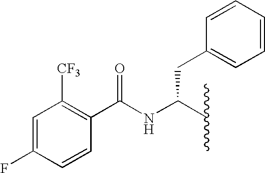 | 3-CF₃ | H |
| 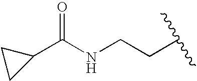 | 3-CF₃ | H |
| 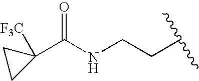 | 3-CF₃ | H |
| 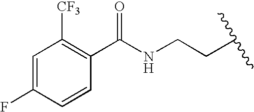 | 3-CF₃ | H |
| 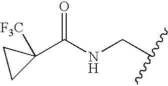 | 3-CF₃ | H |
| 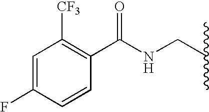 | 3-CF₃ | H |
| 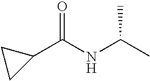 | 3-CF₃ | H |
| 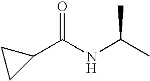 | 3-CF₃ | H |
| 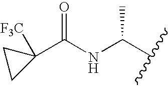 | 3-CF₃ | H |
| 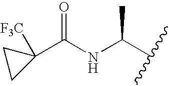 | 3-CF₃ | H |
| 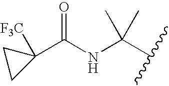 | 3-CF₃ | H |
| 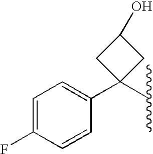 | 3-CF₃ | H |
| 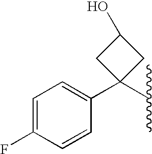 | 3-CF₃ | H |
| 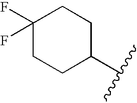 | 3-CF₃ | H |
| 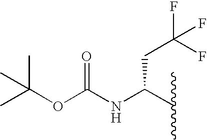 | 3-CF₃ | H |
| 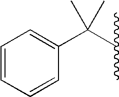 | 3-CF₃ | H |
| 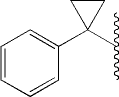 | 3-CF₃ | H |
| 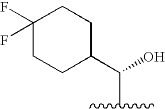 | 3-CF₃ | H |
| 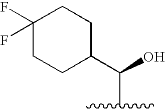 | 3-CF₃ | H |
| (CH₃)₃C—O— | 4-OCF₃ | H |
| 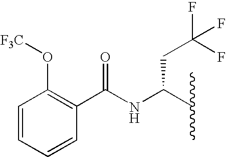 | 3-CF₃ | H |
| 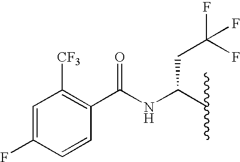 | 3-CF₃ | H |
| 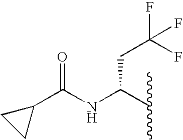 | 3-CF₃ | H |
| 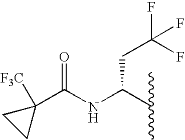 | 3-CF₃ | H |
| 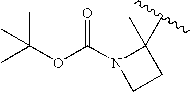 | 3-CF₃ | H |
| 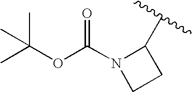 | 3-CF₃ | H |
| 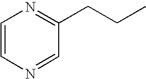 | 3-CF₃ | H |

or a pharmaceutically acceptable salt or an individual diastereomer thereof.

15. The compound of claim 1 of the formula: 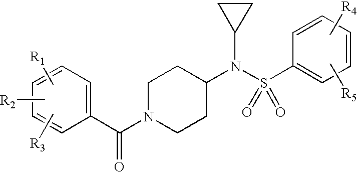
wherein R₁, R₂, R₃, R₄ and R₅ are selected from a single row in the table:

| R₁ | R₂ | R₃ | R₄ | R₅ |
|---|---|---|---|---|
| 3-CF₃ | 5-CF₃ | H | H | H |
| 2-CH₃SO₂ | H | H | 3-CF₃ | H |
| 3-CH₃SO₂ | H | H | 3-CF₃ | H |
| 4-SO₂NH₂ | H | H | 3-CF₃ | H |
| 4-CH₃SO₂ | H | H | 3-CF₃ | H |
| 3-CONH₂ | H | H | 3-CF₃ | H |
| 3-COOCH₃ | H | H | 3-CF₃ | H |
| 4-COOCH₃ | H | H | 3-CF₃ | H |
| 2-CH₃SO₂ | H | H | 4-OCF₃ | H |
| 2-CH₃SO₂ | 5-F | H | 3-CF₃ | H |
| 2-CH₃SO₂ | 4-Cl | H | 3-CF₃ | H |
| 2-CH₃SO₂ | 4-Cl | H | 3-OCF₃ | H |
| 2-CH₃SO₂ | 5-F | H | 3-OCF₃ | H |
| 2-CH₃SO₂ | 6-F | H | 3-CF₃ | H |
| 2-CH₃SO₂ | 4-F | H | 3-CF₃ | H |
| 2-CH₃SO₂ | 4-F | 5-F | 3-CF₃ | H |
| 2-CH₃SO₂ | 6-OCF₃ | H | 3-CF₃ | H |
| 2-CH₃SO₂ | H | H | 2-CF₃ | H |
| 2-CH₃SO₂ | 5-F | H | 2-CF₃ | H |
| 2-CH₃SO₂ | H | H | 2-OCF₃ | H |
| 2-CH₃SO₂ | 5-F | H | 2-OCF₃ | H |
| 2-CH₃CO | H | H | 3-CF₃ | H |
| 2-CH₃SO₂ | 5-F | H | 2-Br | 3-CF₃ |
| 2-CH₃SO₂ | H | H | 2-Cl | 3-CF₃ |
| 2-CH₃SO₂ | 5-F | H | 2-Cl | 3-CF₃ |
| 2-CONH₂ | H | H | 3-CF₃ | H |
| 2-COOCH₃ | H | H | 3-CF₃ | H |
| 2-CONHCH₃ | H | H | 3-CF₃ | H |
| 2-CH₃SO₂ | H | H | 3-CH₃SO₂ | H |
| 2-CH₃SO₂ | 5-F | H | 3-CH₃SO₂ | H |
| 2-CH₃SO₂ | 3-F | H | 3-CF₃ | H |
| 2-CH₃SO₂ | 5-CN | H | 3-CF₃ | H |
| 2-CH₃SO₂ | 5-F | H | 3-CF₃ | 5-CF₃ |
| 2-CH₃SO₂ | 5-F | H | 3-CF₃ | 5-F |

or a pharmaceutically acceptable salt or an individual diastereomer thereof.

16. The compound of claim 1 of the formula: 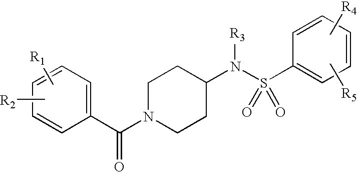
wherein R₁, R₂, R₃, R₄ and R₅ are selected from a single row in the table:

| R₁ | R₂ | R₃ | R₄ | R₅ |
|---|---|---|---|---|
| 3-CF₃ | 4-F | H | 3-CF₃ | H |
| 2-CH₃SO₂ | H | CH₃ | 3-CF₃ | H |
| 2-CH₃SO₂ | H | Ethyl | 3-CF₃ | H |
| 2-OCF₃ | H | 4-(OCH₃)Ph | 3-CF₃ | H |
| 2-OCF₃ | H | H | 3-CF₃ | H |
| 2-CH₃SO₂ | H | H | 3-CF₃ | H |
| 2-CH₃SO₂ | 5-F | H | 3-CF₃ | H |
| 2-CH₃SO₂ | H | Isopropyl | 3-CF₃ | H |
| 2-CH₃SO₂ | 5-F | Isopropyl | 3-CF₃ | H |
| 2-CH₃SO₂ | 4-F | Isopropyl | 3-CF₃ | H |

or a pharmaceutically acceptable salt or an individual diastereomer thereof.

17. A pharmaceutical composition comprising an inert carrier and an effective amount of a compound according to claim 1.

18. A method for treating or preventing chronic or neuropathic pain in a mammalian patient in need thereof comprising administering to said patient a therapeutically effective amount, or a prophylactically effective amount, of a compound according to claim 1, or a pharmaceutically acceptable salt thereof.
